SEQUENCE LISTING (6 sequences)

SEQ ID NO: 1 - DNA, 25 bp, Artificial Sequence
  Description of Artificial Sequence Synthetic primer
cctctcccaatctctaccaggtatc

SEQ ID NO: 2 - DNA, 20 bp, Artificial Sequence
  Description of Artificial Sequence Synthetic primer
aacctctggcctggctgact

SEQ ID NO: 3 - DNA, 18 bp, Artificial Sequence
  Description of Artificial Sequence Synthetic probe
tacagggacgatgacttt

SEQ ID NO: 4 - DNA, 25 bp, Artificial Sequence
  Description of Artificial Sequence Synthetic primer
aactatagagctgccaagtgacaca

SEQ ID NO: 5 - DNA, 16 bp, Artificial Sequence
  Description of Artificial Sequence Synthetic probe
aagcctgctgttgcct

SEQ ID NO: 6 - DNA, 26 bp, Artificial Sequence
  Description of Artificial Sequence Synthetic primer
atttggtacgtgagagatgatatggt

(19) United States
(12) Patent Application Publication
(10) Pub. No.: US 2011/0027771 A1
(43) Pub. Date: Feb. 3, 2011

(54) METHODS AND COMPOSITIONS FOR CELL STABILIZATION

(75) Inventor: David Deng, Mountain View, CA (US)

Correspondence Address: WILSON, SONSINI, GOODRICH & ROSATI, 650 PAGE MILL ROAD, PALO ALTO, CA, 94304-1050, US

(73) Assignee: Artemis Health, Inc., San Carlos, CA (US)

(21) Appl. No.: 12/847,876

(22) Filed: Jul. 30, 2010

Related U.S. Application Data
(60) Provisional application No. 61/230,638, filed on Jul. 31, 2009.

(51) Int. Cl. C12N 5/073; C09K 15/00; C07H 21/04; C12Q 1/02; G01N 33/48; C12Q 1/68; C12M 1/24

(52) U.S. Cl. 435/2; 435/374; 252/397; 536/23.1; 435/29; 436/63; 436/94; 435/6; 435/307.1

(57) ABSTRACT

Fragile cells have value for use in diagnosing many types of conditions. There is a need for compositions that stabilize fragile cells. The stabilization compositions of the provided invention allow for the stabilization, enrichment, and analysis of fragile cells, including fetal cells, circulating tumor cells, and stem cells.

This application claims priority or the benefit under 35 U.S.C. 119 of U.S. provisional application No. 61/230,638 filed Jul. 31, 2009, the contents of which are fully incorporated herein by reference.

### FIELD OF THE INVENTION

The present invention relates to compositions and method for the stabilization, enrichment, and analysis of fragile cells, including fetal cells, circulating tumor cells, and stem cells.

### BACKGROUND OF THE INVENTION

Fragile cells can be used in tests to diagnose the presence or absence of disease. For example, fragile fetal cells isolated from maternal samples can be used for prenatal diagnostics, and fragile circulating tumor cells can be useful for diagnosing various patient conditions. The means by which fragile cells are handled can play a role in various tests. Fragile cells are often rare, and enrichment of these cells can aid analysis of these cells. Furthermore, diagnostic tests performed using these cells can take place hours or days after a sample containing the cells is retrieved. Thus, means for maintaining the integrity of a rare cell through one or more enrichment steps and/or over extended periods of time (hours or days) can play a role in the ability to analyze the cells and perform diagnostic tests. To facilitate enrichment and analysis of fragile cells, there is a need for improved compositions, methods, and kits for stabilizing fragile cells (e.g., fetal cells, circulating tumor cells, and stem cells) in vitro. Compositions that stabilize fragile cells can also be used to stabilize other cell types.

### SUMMARY OF THE INVENTION

In one aspect, a stabilization composition is provided capable of maintaining at least 50% of fetal cells in a blood sample intact for at least 6 hr. In another aspect, a stabilization composition is provided capable of maintaining at least 50% of fetal nucleated red blood cells intact for at least 6 hr. In one embodiment, the composition is capable of maintaining at least 50% of fetal nucleated red blood cells intact for at least 12 hr, at least 24 hr, at least 48 hr, at least 72 hr, or at least 96 hr. In another embodiment, a composition is provided comprising one or more isolated fetal cells in a stabilization composition. In another embodiment, the composition is a solution.

In another aspect, a stabilization composition is provided including four or more anticoagulants and two or more antioxidants. In another aspect, the stabilization composition further includes one or more of the following: one or more energy sources; one or more cell membrane stabilizers; and one or more cross-linking agents.

In another aspect, a stabilization composition is provided including two or more antioxidants and one or more cross-linking agents.

In one embodiment, the stabilization composition further includes one or more of the following: one or more anticoagulants; one or more energy sources; and one or more cell membrane stabilizers.

In another aspect, a stabilization composition is provided including: glycine, NAC, glutamine and D-Mannitol and optionally one or more anticoagulants, cell membrane stabilizers, or energy sources.

In one embodiment, the composition does not include (i) formaldehyde or (ii) an agent that slows cell metabolism.

In one embodiment, the composition does not include (i) potassium dichromate or (ii) a cell membrane stabilizing agent.

In one embodiment, the anticoagulant comprises at least one antiplatelet drug.

In one embodiment, the at least one antiplatelet drug is selected from the group consisting of theophylline and dipyridamole.

In one embodiment, the anticoagulant comprises one or more of lithium heparin, sodium heparin, citrate heparin, ammonia heparin, sodium citrate, dipyridamole, theophylline, adenine, adenosine, Warfarin, acenocoumarol, phenindione, low molecular weight heparin, idraparinux, fondaparinux, argatroban, lepirudin, bivalirudin, and dabigatran.

In one embodiment, the energy source includes glucose, lactose, fructose, or galactose.

In one embodiment, the antioxidant includes glycine, n-acetyl-L-cysteine, glutamine, D-Mannitol, vitamin C (ascorbic acid), vitamin E (tocopherols and tocotrienols), green tea, ferulic acid, reduced glutathione, melatonin, resveratrol, vitamin A (palmitate), beta carotene, vitamin D-3 (cholecalciferol), selenium (1-seleno methionine), BHA, or BHT.

In one embodiment, the cell membrane stabilizer includes one or more of potassium dichromate, cadmium chloride, or lithium chloride aldehydes, urea formaldehyde, phenol formaldehyde, DMAE (dimethylaminoethanol), cholesterol, cholesterol derivatives, high concentrations of magnesium, vitamin E, and vitamin E derivatives, calcium, calcium gluconate, taurine, niacin, hydroxylamine derivatives, bimoclomol, sucrose, astaxanthin, glucose, amitriptyline, isomer A hopane tetral phenylacetate, isomer B hopane tetral phenylacetate, citicoline, inositol, vitamin B, vitamin B complex, cholesterol hemisuccinate, sorbitol, calcium, coenzyme Q, ubiquinone, vitamin K, vitamin K complex, menaquinone, zonegran, zinc, ginkgo biloba extract, diphenylhydantoin, perftoran, polyvinylpyrrolidone, phosphatidylserine, tegretol, PABA, disodium cromglycate, nedocromil sodium, phenyloin, zinc citrate, mexitil, dilantin, sodium hyaluronate, or polaxamer 188.

In one embodiment, the cross-linking agent includes one or more of formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfydryl addition or disulfide reduction, carbohydrate reactive crosslinkers, carboxyl reactive crosslinkers, photoreactive crosslinkers, cleavable crosslinkers, AEDP, APG, BASED, BM(PEO)3, BM(PEO)4, BMB, BMDB, BMH, BMOE, BS3, BSOCOES, DFDNB, DMA, DMP, DMS, DPDPB, DSG, DSP, DSS, DST, DTBP, DTME, DTSSP, EGS, HBVS, sulfo-BSOCOES, Sulfo-DST, or Sulfo-EGS.

In one embodiment, the composition further includes one or more of PEG-200, PEG-300, PEG-400, PEG-600, PEG-1000, PEG-1450, PEG-3350, PEG-4000, PEG-6000, PEG-8000, PEG-20,000, imidazolidinyl urea, diazolidinyl urea, calcium propionate, sodium nitrate, sodium nitrite, sulfites, sulfur dioxide, sodium bisulfite, potassium hydrogen sulfite, disodium EDTA, ethanol, or methylchloroisothiazolinone.

In one embodiment, the composition further comprises a buffer.

In one embodiment, the buffer comprises one or more of phosphate buffered saline (PBS), TAPS, Bicine, Tris, Tricine, HEPES, TES, MOPS, PIPES, Cacodylate, or MES.

In another aspect, a method for stabilizing a cell or cellular component is provided comprising contacting said cell or cellular component with a composition of any one of claims 6-10.

In another embodiment, the cellular component is cell-free DNA. In another embodiment, the cell is a fetal cell in a maternal blood sample.

In another aspect, a method for diagnosing a fetal condition is provided comprising: contacting a maternal blood sample with a stabilization composition of any one of claims 6-10; and analyzing one or more cells or cellular components from said sample to diagnosis said fetal condition.

In another embodiment, the method further includes enriching fetal cells from said sample using size-based separation, selective red blood cell lysis, or density gradient centrifugation.

In another embodiment, the method further includes contacting the sample with a lysis reagent that selectively lysis enucleated red blood cells over nucleated red blood cells.

In another embodiment, the method further includes performing an antibody-based enrichment step.

In another embodiment, the analyzing comprises performing fluorescent in-situ hybridization on DNA from said one or more cells or cellular components from said sample.

In another embodiment, the fetal condition comprises fetal aneuploidy. In another embodiment, the aneuploidy includes trisomy. In another embodiment, the trisomy includes trisomy 13, trisomy 18, or trisomy 21.

In another embodiment, the cellular component includes cell-free DNA. In another embodiment, the analyzing includes DNA sequencing. In another embodiment, the DNA sequencing includes sequencing DNA from a first genomic region suspected of being trisomic and a second genomic region suspected of being aneuploid.

In another embodiment, the analyzing comprises digital PCR. In another embodiment, the cell is a fetal nucleated red blood cell.

In another aspect, a test tube or syringe with a plug or a solution is provided including a stabilization solution capable of maintaining at least 50% of fetal cells in a blood sample intact for at least 6 hr.

In another aspect, a test tube or syringe with a plug or a solution is provided including a stabilization solution capable of maintaining at least 50% of fetal nucleated red blood cells in a blood sample intact for at least 6 hr.

In one embodiment, the composition is capable of maintaining at least 50% of fetal nucleated red blood cells intact for at least 12 hr, at least 24 hr, at least 48 hr, at least 72 hr, or at least 96 hr.

In another aspect, a test tube or syringe with a plug or a solution is provided including a stabilization solution comprising: four or more anticoagulants; and two or more antioxidants.

In one embodiment, the test tube or syringe further comprise one or more of the following: one or more energy sources; one or more cell membrane stabilizers; and one or more cross-linking agents.

In another aspect, a test tube or syringe with a plug or a solution comprising a stabilization solution is provided including: two or more antioxidants; and one or more cross-linking agents.

In one embodiment, the test tube or syringe further include one or more of the following: one or more anticoagulants; one or more energy sources; and one or more cell membrane stabilizers.

In another aspect, a test tube or syringe with a plug or a solution is provided including a stabilization solution including: glycine, NAC, glutamine and D-Mannitol and optionally one or more anticoagulants, cell membrane stabilizers, or energy sources.

In another embodiment, a kit including a test tube or syringe is provided further including instructional material and materials for shipping a blood sample.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates that fetal cells display higher stability in a composition containing heparin compared to a composition containing EDTA.

FIG. 2 illustrates that more fetal cells are stabilized over 6 hr in Composition C than in a solution that lacks Composition C.

FIG. 3 depicts numbers of cells equivalents in 10 mL blood at 1, 24, 48, 72, and 96 hr after collection in Composition A.

FIG. 4 shows the number of cell equivalents (CE) from 10 mL whole blood at 24 hr.

FIG. 5 depicts a rating summary of fetal cell stabilization compositions.

FIG. 6 demonstrates that more fetal cells were observed in 8 out of 11 samples that contained ACD+Composition D relative to samples that contained ACD+CytoCheck®. The samples were enriched by density gradient centrifugation (“DGC”) or size-based cell separation on a two-dimensional array of obstacles (“CSM”).

FIG. 7 illustrates blood cell morphology in ACD+Composition D at 76 hrs for two different samples.

FIG. 8 depicts a procedure for testing for fetal cell recovery after size-based cell separation.

FIGS. 9A-9D illustrate embodiments of a size-based separation module.

FIGS. 10A-10D show a schematic of a device used to separate fetal nucleated red blood cells from maternal blood.

FIGS. 11A-B show the total white blood cell (WBC) count and red blood cell count (RBC), respectively, before and after treatment of blood samples with lytic agent HYL-250.

FIG. 12 illustrates the effect of Composition Q on the retention of fetal cells in maternal blood during lysis of RBCs. FIG. 12 illustrates the use of Y loci 5 kb apart on the male specific gene RPS4Y2 for fetal cell enumeration by digital PCR.

FIG. 13 shows fetal cells identified by immunocytochemistry and DNA FISH following enrichment by RBC lysis and CD71 antibody-based enrichment.

### DETAILED DESCRIPTION OF THE INVENTION

### I. Overview

In general, the provided invention includes compositions for stabilizing cells. The cells that can be stabilized by the compositions of the provided invention include rare cells, for example, fetal cells in maternal blood, circulating tumor cells, circulating epithelial cells, circulating endothelial cells, or stem cells. The rare cells can be in a fluid containing a mixture of rare cells and non-rare cells (e.g., blood). The stabilization compositions of the provided invention can stabilize non-rare cells (e.g., maternal cells in a maternal blood sample). The provided invention also includes methods for using cell stabilization compositions for enriching rare cells, for example circulating tumor cells (CTCs), fetal cells (e.g., in maternal blood), circulating epithelial cells, circulating endothelial cells, and stem cells. The provided invention also includes methods for diagnostic assays (e.g., prenatal diagnostics) that include using cells or cellular components (e.g., cell-free DNA) that have been contacted by a stabilization composition.

In one embodiment, a stabilization composition is provided that can stabilize a cell. Markers of stabilization can include, for example, an intact cell membrane, viability, culturability, preservation of antigen expression, and a lack of change of cell morphology.

The compositions of the provided invention can stabilize at least 50%, at least 55%, at least 60%, at least 65%, at least 70%, at least 75%, at least 80%, at least 85%, at least 90%, at least 95%, or at least 100% of cells (e.g., rare cells) in a sample for at least 1 hr, at least 2 hr, at least 3 hr, at least 4 hr, at least 5 hr, at least 6 hr, at least 7 hr, at least 8 hr, at least 9 hr, at least 10 hr, at least 11 hr, at least 12 hr, at least 24 hr, at least 48 hr, at least 72 hr, or at least 96 hr. Examples of rare cells include circulating tumor cells (CTCs), fetal cells, circulating epithelial cells, circulating endothelial cells, and stem cells. Examples of samples include a mixed cell sample, blood sample, maternal blood sample, a sample containing cell-free DNA, and a sample with cells or cellular components obtained after a purification or enrichment step. A sample can contain a mixture of rare and non-rare cells.

The compositions of the provided invention can stabilize cells when used at about 4° C., about 10° C., about 15° C., about 20° C., about 21° C., about 22° C., about 23° C., about 24° C., about 25° C., about 26° C., about 27° C., about 28° C., about 29° C., and about 30° C. The compositions of the provided invention can stabilize cells when used at room temperature (i.e. approximately 24 to 25.5° C.).

The compositions of the provided invention can have the property of not affecting immuno-based cell enrichment procedures or immuno-based cell identification procedures. The compositions can facilitate cell separation procedures, e.g., size-based separation through an array of two-dimensional obstacles.

In one embodiment, a method for stabilizing a cell or cellular component is provided that includes contacting a cell or cellular component with a composition of the provided invention. In one embodiment, a stabilization composition is provided that can maintain at least 50%, at least 55%, at least 60%, at least 65%, at least 70%, at least 75%, at least 80%, at least 85%, at least 90%, at least 95%, or at least 100% of fetal cells from a maternal blood sample intact for at least 6 hrs, at least 12 hr, at least 24 hr, at least 48 hr, at least 72 hr, or at least 96 hr. In another embodiment, a stabilization composition is provided that can maintain at least 50%, at least 55%, at least 60%, at least 65%, at least 70%, at least 75%, at least 80%, at least 85%, at least 90%, at least 95%, or at least 100% of fetal nucleated red blood cells (fnRBCs) from a maternal blood sample intact for at least 6 hrs, at least 12 hr, at least 24 hr, at least 48 hr, at least 72 hr, or at least 96 hr.

### II. Stabilization Composition Components

The compositions, methods, and kits of the provided invention can include anticoagulants (which can include platelet aggregation inhibitors), energy sources, antioxidants, cell membrane stabilizers, cross-linking agents, or other components.

In one embodiment, a stabilization composition is provided capable of maintaining a least 50% cells intact for at least 6 hours, and can lack formaldehyde or an agent that slows cell metabolism. The composition can lack potassium dichromate or a cell membrane stabilizing agent.

In another one embodiment, a stabilization composition can include four or more anticoagulants and two or more antioxidants, and can also include one or more of the following: one or more energy sources; one or more cell membrane stabilizers; and one or more cross-linking agents. The composition can lack potassium dichromate or a cell membrane stabilizing agent.

In another embodiment, a stabilization composition is provided that can include two or more antioxidants and one or more cross-linking agents, and can also include one or more of the following: one or more anticoagulants, one or more energy sources, and one or more cell membrane stabilizers.

In another embodiment, a stabilization composition is provided that can include glycine, N-acetyl-L-cysteine (NAC), glutamine, and D-Mannitol, and optionally one or more anticoagulants, cell membrane stabilizers, or energy sources. The composition can lack formaldehyde or an agent that slows cell metabolism.

In another embodiment, a stabilization composition is provided that can include at least two anticoagulants, of which at least one is an antiplatelet drug, at least one energy source, at least two antioxidants, at least one cell membrane stabilizer, and at least one cross linking agent.

In another embodiment, a stabilization composition is provided that can include at least one buffer, at least one inorganic salt, at least one fixative, at least one cell membrane stabilizer, and at least two anticoagulants.

In another embodiment, a stabilization composition is provided that can include at least three anticoagulants, at least four antioxidants, at least two cell membrane stabilizing agents, at least one buffer, at least one cross-linking agent, at least one red blood cell lysis agent, at least one inorganic salt, and at least two other additives.

In another embodiment, a stabilization composition is provided that can include at least four anticoagulants, at least two antioxidants, at least one cell membrane stabilizing agent, at least one buffer, at least two red blood cell lysis agents, and at least two other additives.

In another embodiment, a stabilization composition is provided that can include at least four anticoagulants, of which at least three are antiplatelet drugs, at least three antioxidants, and least one buffer, at least three cell membrane stabilizers, at least one cross linking agent, at two inorganic salts, and at least one additive.

In another embodiment, a stabilization composition is provided that can include at least one energy source, at least one anticoagulant, at least two antioxidants, and at least one buffer.

A. Anticoagulants

Suitable anticoagulants for use in the compositions, methods, and kits of the provided invention can include, for example, a) inhibitors of clotting factor synthesis, b) inhibitors of thrombin, and c) antiplatelet drugs.

Examples of inhibitors of clotting factor synthesis include warfarin (Coumadin), a derivative of coumarin. Other derivatives of coumarin include, for example, phenprocoumon (Marcoumar) and acenocoumarol (Sintrom). Coumatetralyl is an anticoagulant of the warfarin type. Dicoumarol (or dicumarol) functions as a Vitamin K antagonist (similar to warfarin), preventing the formation of prothrombin. Pindone inhibits Vitamin K dependent clotting factors.

Two types of direct thrombin inhibitors (DTIs) are bivalent DTIs and univalent DTIs. Bivalent DTIs include hirudin, bivalirudin (Angiomax), lepirudin (Refludan), and desirudin. Univalent DTIs include argatroban, melagatran (and its prodrug ximelagatran), and dabigatran. Other examples of inhibitors of thrombin include heparin (e.g., lithium heparin, sodium heparin, citrate heparin, ammonia heparin, low molecular weight heparin), which can bind and activate the enzyme inhibitor antithrombin (AT), which then inactivates thrombin. Dalteparin is a low molecular weight heparin. Enoxaparin (Lovenox or Clexane) is a low molecular weight heparin. ATryn® is the brand name of a recombinant form of antithrombin manufactured by GTC Biotherapeutics.

Examples of antiplatelet drugs include cyclooxygenase inhibitors (e.g., aspirin), adenosine diphosphate (ADP) receptor inhibitors (e.g., ticlopidine (Ticlid), clopidogrel (Plavix), and theophylline (dimethylxanthine)), phophodiesterase inhibitors (e.g., cilostazol (Pletal)), glycoprotein IIB/IIIA receptor antagonists (e.g., murine-human chimeric antibodies (e.g., abciximab (ReoPro)), synthetic non-peptides (e.g., tirofiban (Aggrastat)), synthetic peptides (e.g., eptifibatide (Integrilin) and defibrotide), and adenosine reuptake inhibitors (e.g., dipyridamole (Persantine)). Adenosine can inhibit platelet activation via adenosine receptors.

Some anticoagulants can function by binding calcium ions, for example, ethylenediaminetetraacetic acid (EDTA), citrate (e.g., sodium citrate; ACD, or Anticoagulant Citrate Dextrose Solution, or acid-citrate-dextrose; citric acid, sodium citrate, and dextrose in water), and oxalate.

Other anticoagulants include brodifacoum (which inhibits the enzyme Vitamin K epoxide reductase), phenindione (Vitamin K antagonist), idraparinux (which blocks coagulation Factor Xa), fondaparinux (Arixtra), adenine, anisindione (Miradon), apixaban (which inhibits coagulation Factor Xa), ardeparin sodium (Normiflo), certoparin, danaparoid sodium (Orgaran, which inhibits activated Factor Xa), defibrotide, hementin, lonomia, nafamostat, otamixaban (which inhibits Factor Xa), rivaroxaban (Xarelto; which is a direct inhibitor of coagulation Factor Xa), and tioclomarol (a Vitamin K antagonist),

Draculin can inhibit coagulation factors IX (IXa) and X (Xa).

In one embodiment, a stabilization composition is provided comprising at least three or four anticoagulants. In another embodiment, a stabilization composition is provided comprising at least three or four anticoagulants, of which at least one or two anticoagulants is an antiplatelet drug.

B. Energy Sources

Suitable energy sources for use in the compositions, methods, and kits of the provided invention can include, for example, glucose, fructose, galactose, mannose, lactose, or maltose. Adenine and adenosine can be used to provide energy by being convertible to ATP. In one embodiment, a stabilization composition is provided comprising at least one energy source.

C. Antioxidants

Suitable antioxidants for use in the compositions, methods, and kits of the provided invention can include, for example, amino acids (e.g., glycine, histidine, tyrosine, tryptophan, glutamine) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl (n-acetyl-L-cysteine (NAC)), methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents, (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C (ascorbic acid) and derivatives (for example ascorbyl palmitate, magnesium ascorbyl phosphate, ascorbyl acetate), tocotrienols, tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, d-mannitol, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide), green tea, reduced melatonin, resveratrol, dipyridamole, vitamin D-3 (cholecalciferol), BHA, and BHT. Suitable antioxidants are described in U.S. Patent Application Publication No. 20090098072. Glycine, N-acetyl-L-cysteine, and glutamine are glutathione (GSH) precursor amino acids.

In one embodiment, a stabilization composition is provided comprising at least one, two or three antioxidants.

D. Cell Membrane Stabilizers

Suitable cell membrane stabilizers that can be used in the methods, compositions, and kits of the provided invention can include, for example, aldehydes, urea formaldehyde, phenol formaldehyde, DMAE (dimethylaminoethanol), cholesterol, cholesterol derivatives, high concentrations of magnesium, vitamin E, and vitamin E derivatives, calcium, calcium gluconate, taurine, niacin, hydroxylamine derivatives, bimoclomol, sucrose, astaxanthin, glucose, amitriptyline, isomer A hopane tetral phenylacetate, isomer B hopane tetral phenylacetate, citicoline, inositol, vitamin B, vitamin B complex, cholesterol hemisuccinate, sorbitol, calcium, coenzyme Q, ubiquinone, vitamin K, vitamin K complex, menaquinone, zonegran, zinc, ginkgo biloba extract, diphenylhydantoin, perftoran, polyvinylpyrrolidone, phosphatidylserine, tegretol, PABA, disodium cromglycate, nedocromil sodium, phenyloin, zinc citrate, mexitil, dilantin, sodium hyaluronate, polaxamer 188, potassium dichromate, cadmium chloride, lithium chloride, adenine/adenosine, dipyridamole, sodium citrate. Suitable cell membrane stabilizers are also described in U.S. Pat. No. 7,332,277. Other suitable cell membrane stabilizers include, for example, a monosaccaride (e.g., glucose, fructose), a sugar alcohol (e.g., sorbitol, inositol), a disaccharide (e.g., sucrose, trehalose, lactose, maltose), a trisaccharide (e.g., raffinose), a oligosaccharide (e.g., cycloinulohexaose), a polysaccharide (e.g., ficoll, or dextran), or a polymer (e.g., poly-vinyl-pyrrolidone, polyethyleneglycol), as described in U.S. Patent Application No. 20050048648.

In one embodiment, a stabilization composition is provided comprising at least one cell membrane stabilizer.

E. Cross-Linking Agents

Suitable cross-linking agent that can be used in the methods, compositions, and kits of the provided invention can include, for example, formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfydryl addition or disulfide reduction, carbohydrate reactive crosslinkers, carboxyl reactive crosslinkers, photoreactive crosslinkers, cleavable crosslinkers, AEDP, APG, BASED, BM(PEO)3, BM(PEO)4, BMB, BMDB, BMH, BMOE, BS3, BSOCOES, DFDNB, DMA, DMP, DMS, DPDPB, DSG, DSP, DSS, DST, DTBP, DTME, DTSSP, EGS, HBVS, sulfo-BSOCOES, Sulfo-DST, or Sulfo-EGS. Additional suitable cross-linkers include succinimidylacetylthioacetate (SATA); succinimidyl trans-4-(maleimidylmethyl) cyclohexane-1-carboxylate (SMCC); succinimidyl 3-(2-pyridyldithio)-propionate (SPDP); N-((2-pyridyldithio)ethyl)-4-azidosalicylamide (PEAS; AET); 4-azido-2,3,5,6-tetrafluorobenzoic acid, succinimidyl ester (ATFB, SE); 4-azido-2,3,5,6-tetrafluorobenzoic acid, STP ester, sodium salt (ATFB, STP ester); 4-azido-2,3,5,6-tetrafluorobenzyl amine, hydrochloride; benzophenone-4-isothiocyanate; benzophenone-4-maleimide; 4-benzoylbenzoic acid, succinimidyl ester; Disuccinimidylsuberate (DSS); Dithiobis(succinimidylpropionate (DSP); 3,3′-Dithiobis(sulfosuccinimidylpropionate) (DTSSP); Bis[2-(sulfosuccinimdooxycarbonyloxy)ethyl]sulfone (BSOCOES); Disulfosuccinimdyltartrate (SULFO DST); Disuccinimdyltartrate (DST); Ethylene glycolbis(succinimidylsuccinate) (EGS); Ethylene glycolbis(sulfosuccinimidylsuccinate) (SULFO-EGS); 1,2-Di[3′-(2′-pyridyldithio)propionamido]butane (DPDPB); Bis(sulfosuccinimdyl)suberate (BSSS); Succinimdyl-4-(p-maleimidophenyl)butyrate (SMPB); Sulfosuccinimdyl-4-(p-maleimidophenyl)butyrate (SULFO SMPB); 3-Maleimidobenzoyl-N-hydroxysuccinimide ester (MBS); 3-Maleimidobenzoyl-N-hydroxysulfosuccinimide ester (SULFO MBS); N-Succinimidyl(4-iodacetyl)aminobenzoate (SLAB); N-Sulfosuccinimidyl(4-iodacetyl)aminobenzoate (SULFO SLAB); Succinimidyl-4-(N-maleimidomethyl)cyclohexane-1-carboxylate (SMCC); Sulfosuccinimidyl-4-(N-maleimidomethyl)cyclohexane-1-carboxylate (SULFO SMCC); Succinimidyl-6-[3-(2-pyridyldithio)propionamido)hexanoate (NHS LC SPDP); Sulfosuccinimidyl-6-[3-(2-pyridyldithio)propionamido)hexanoate (SULFO NHS LS SPDP); N-Succinimdyl-3-(2-pyridyldithio)propionate (SPDP); N-Hydroxysuccinimidylbromoacetate (NHS BROMOACETATE); N-Hydroxysuccinimidyliodoacetate (NHS IODOACETATE); 4-(N-Maleimidophenyl)butyric acid hydrazide hydrochloride (MPBH); 4-(N-Maleimidomethyl)cyclohexane-1-carboxylic acid hydrazide hydrochloride (MCCH); m-Maleimidobenzoic acid hydrazidehydrochloride (MBH); N-(epsilon-Maleimidocaproyloxy)sulfosuccinimide (SULFO EMCS); N-(epsilon-Maleimidocaproyloxy)succinimide (EMCS); N-(p-Maleimidophenyl)isocyanate (PMPI); N-(kappa-Maleimidoundecanoic acid) hydrazide (KMUH); Succinimidyl-4-(N-maleimidomethyl)-cyclohexane-1-carboxy(6-amidocaproate) (LC SMCC); N-(gamma-Maleimidobutryloxy)sulfosuccinimide ester (SULFO GMBS); Succinimidyl-6-(beta-maleimidopropionamidohexanoate (SMPH) N-(kappa-Maleimidoundecanoyloxy)sulfosuccinimide ester (SULFO KMUS); N-(gamma-Maleimidobutyrloxy)succinimide (GMBS); Dimethyladipimidate hydrochloride (DMA); Dimethylpimelimidate hydrochloride (DMP); Dimethylsuberimidate hydrochloride (DMS); Methyl-p-hydroxybenzimidate hydrochloride, 98% Amine Reactive (MHBH (Wood's Reagent)); Bis[sulfosuccinimidyl]suberate (BS3); Bis[2-(succinimidooxycarbonyloxy)ethyl]sulfone (BSOCOES); Disuccinimidyl glutarate (DSG); DSP (Lomant's Reagent); 1,5-Difluoro-2,4-dinitrobenzene (DFDNB); Dithiobis[succinimidylpropionate (DTBP); Bis-[b-(4-Azidosalicylamido)ethyl]disulfide, Sulfhydryl Reactive (BASED); BM[PEO]₃(1,8-bis-Maleimidotriethyleneglycol (BM[PEO]₃); BM[PEO]₄(1,11-bis-Maleimidotetraethyleneglycol (BM[PEO]₄); 1,4-bis-Maleimidobutane (BMB); 1,4-bis-Maleimidyl-2,3-dihydroxybutane (BMDB); Bis-Maleimidohexane (BMH); 1,4-Di-[3;-(2′-pyridyldithio)-propionamido]butane (DPDPB); Dithio-bis-maleimidoethane (DTME); 1,6-Hexane-bis-vinylsulfone (HBVS); p-Azidobenzoyl hydrazide (ABH); N-[a-Maleimidoacetoxy)succinimide ester (AMAS); N-[4-(p-Azidosalicylamido)butyl]-3′-(2′pyridyldithio)propionamide (APDP); N-[β-Maleimidopropyloxy]succinimide ester (BMPS); 4-(N-M-Maleimidomethyl)cyclohexane-1-carboxylic acid hydrazide hydrochloride (MCCH); m-Maleimidobenzoic acid hydrazidehydrochloride (MBH); N-(epsilon-Maleimidocaproyloxy)sulfosuccinimide (SULFO EMCS); N-(epsilon-Maleimidocaproyloxy)succinimide (EMCS); N-e-Maleimidocaproic acid (EMCA); N-e-Maleimidocaproyloxy]succinimide ester (EMCS); N-[g-Maleimidobutyryloxy]succinimide ester (GMBS); N-k-Maleimidoundecanoic acid (KMUA); Succinimidyl-4-(N-Maleimidomethyl)cyclohexane-1-carboxy-(6-amidocaproate) (LC-SMCC); Succinimidyl 6-(3-[2-pyridyldithio]-propionamido)hexanoate (LC-SPDP); m-Maleimidobenzoyl-N-hydroxysuccinimide ester (MBS); Succinimidyl 3-[bromoacetamido]propionate (SBAP); N-Succinimidyl iodoacetate (SIA); N-Succinimidyl[4-iodoacetyl]aminobenzoate (SIAB); Succinimidyl 4-[N-maleimidomethyl]cyclohexane-1-carboxylate (SMCC); Succinimidyl 4-[p-maleimidophenyl]butyrate (SMPB); Succinimidyl-6-[β-maleimidopropionamido]hexanoate (SMPH); 4-Succinimidyloxycarbonyl-methyl-a-[2-pyridyldithio]toluene (SMPT); N-Succinimidyl 3-[2-pyridyldithio]-propioamido (SPDP); N-e-Maleimidocaproyloxy]sulfosuccinimide ester (Sulfo-EMCS); N-[g-Maleimidobutyryloxy]sulfosuccinimide ester (Sulfo-GMBS); N-[k-Maleimidoundecanoyloxy]sulfosuccinimide ester (Sulfo-KMUS); 4-Sulfosuccinimidyl-6-methyl-a-(2-pyridyldithio)toluamido]hexanoate (Sulfo-LC-SMPT); Sulfosuccinimidyl 6-(3′-[2-pyridyldithio]-propionamido)hexanoate (Sulfo-LC-SPDP); m-Maleimidobenzoyl-N-hydroxysulfosuccinimide ester (Sulfo-MBS); N-Sulfosuccinimidyl[4-iodoacetyl]aminobenzoate (Sulfo-SIAB); Sulfosuccinimidyl 4-[N-maleimidomethyl]cyclohexane-1-carboxylate (Sulfo-SMCC); Sulfosuccinimidyl-4-(P-Maleimidophenyl) Butyrate (Sulfo-SMPB); N-5-Azido-2-nitrobenzoyloxysuccinimide (ANB-NOS); Methyl N-succinimidyl adipate (MSA); N-Hydroxysuccinimidyl-4-azidosalicylic acid (NHS-ASA); N-Succinimidyl(4-azidophenyl)-1,3′-dithiopropionate (SADP); Sulfosuccinimidyl 2-[7-amino-4-methylcoumarin-3-acetamido]ethyl-1,3′dithiopropionate (SAED); Sulfosuccinimidyl 2[m-azido-o-nitrobenzamido]-ethyl-1,3′-dithiopropionate (SAND); N-Succinimidyl-6-[4′-azido-2′-nitrophenylamino]hexanoate (SANPAH); Sulfosuccinimidyl-2-[p-azidosalicylamido]ethyl-1,3′dithiopropionate (SASD); Sulfosuccinimidyl-[perfluoroazidobenzamido]ethyl-1,3′-dithiopropionate (SFAD); N-Hydroxysulfosuccinimidyl-4-azidobenzoate (Sulfo-HSAB); N-(epsilon-Maleimidocaproyloxy)succinimide (EMCS); Sulfosuccinimidyl[4-azidosalicylamido]-hexanoate (Sulfo-NHS-LC-ASA); N-Sulfosuccinimidyl(4-azidophenyl)-1,3′-dithiopropionate (Sulfo-SADP); N-Sulfosuccinimidyl-6-[4′-azido-2′-nitrophenylamino]hexanoate (Sulfo-SANPAH); p-Azidophenyl glyoxal monohydrate (APG); N-β-Maleimidopropionic acid (BMPA); N-Succinimidyl-S-acetylthiopropionate (SATP); 4-(4-N-Maleimidophenyl)butyric acid hydrazide hydrochloride (MPBH); 3-(2-Pyridyldithio)propionyl hydrazide (PDPH); N-[β-Maleimidopropionic acid]hydrazide-TFA (BMPH); N-e-Maleimidocaproic acid]hydrazide (EMCH); N-[k-Maleimidoundecanoic acid]hydrazide (KMUH); and N-[p-Maleimidophenyl]isocyanate (PMPI), or TFCS. Suitable cross-linking agents are also described in U.S. Pat. No. 7,332,277.

A cross-linking agent can be used with a metal salt. The ratio of the cross-linking agent to the metal salt can be about 0.4, about 0.44, about 0.5, about 0.54, about 0.6, about 0.64, about 0.7, about 0.74, about 0.8, about 0.84, about 0.9, about 1.0, about 1.1, about 1.2, about 1.3, about 1.4, about 1.5, about 1.6, about 1.7, about 1.8, about 1.9, or about 2.0. The ratio of the cross-linking agent to the metal salt can be at least 0.4, at least 0.44, at least 0.5, at least 0.54, at least 0.6, at least 0.64, at least 0.7, at least 0.74, at least 0.8, at least 0.84, at least 0.9, at least 1.0, at least 1.1, at least 1.2, at least 1.3, at least 1.4, at least 1.5, at least 1.6, at least 1.7, at least 1.8, at least 1.9, or at least 2.0. In one embodiment, the ratio of formaldehyde to dichromate is about 0.4, about 0.44, about 0.5, about 0.54, about 0.6, about 0.64, about 0.7, about 0.74, about 0.8, about 0.84, about 0.9, about 0.94, about 1.0, about 1.1, about 1.2, about 1.3, about 1.4, about 1.5, about 1.6, about 1.7, about 1.8, about 1.9, or about 2.0. In one embodiment, the ratio of formaldehyde to dichromate is at least 0.4, at least 0.44, at least 0.5, at least 0.54, at least 0.6, at least 0.64, at least 0.7, at least 0.74, at least 0.8, at least 0.84, at least 0.9, at least 0.94, at least 1.0, at least 1.1, at least 1.2, at least 1.3, at least 1.4, at least 1.5, at least 1.6, at least 1.7, at least 1.8, at least 1.9, or at least 2.0.

In one embodiment, a stabilization composition is provided comprising at least one cross-linking agent. In one embodiment, a stabilization composition is provided comprising at least one cross-linking agent and at least one metal salt.

F. Buffers

The compositions of the provided invention can include one or more buffers. Suitable buffers for use in the compositions, methods, and kits of the provided invention can include, for example, one or more of phosphate buffered saline (PBS), TAPS, Bicine, Tris, Tricine, HEPES, TES, MOPS, PIPES, Cacodylate, MES, Bis-Tris, ADA, aces, MOPSO, Bis-Tris-Propane, BES, DIPSO, MOBS, TAPSO, Trizma, HEPPSO, POPSO, TEA, EPPS, Gly-Gly, Bicine, HEPBS, AMPD, TABS, AMPSO, CHES, CAPSO, AMP, CAPS, or CABS. The buffer can be a phosphate buffer, a citrate/citric acid buffer, an acetate/acetic acid buffer, an imidazole (glycoxaline) buffer, or a carbonate/bicarbonate buffer, The pH of the compositions of the provided invention at 25° C. can be, e.g., pH 5-12, pH 6-11, pH 6-10, pH 6-9, pH 6-8, pH 6-7, pH 7-8, pH 7-9, pH 7-10, or about pH 5.5, about pH 6.0, about pH 6.5, about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.5, about pH 9.0, about pH 9.5, about pH 10, or about pH 10.5.

In one embodiment, a stabilization composition is provided comprising at least one buffer.

G. Fixatives

The compositions of the provided invention can include one or more fixatives. Suitable fixatives for use in the compositions, methods, and kits of the provided invention include formaldehyde, paraformaldehyde, glutaraldehyde, acrolein, glyoxal, malonaldehyde, diacetyl, polyaldehydes, carbodiimides, diisocyanates, diazonium compounds, diimido esters, diethylpyrocarbonate, maleimides, benzoquinone, and metallic ions, Dinitrobenzaldehyde, Dinitrobenzene sulfonic acids, or Dinitrobenzoic acids. In another embodiment the fixative is a Dinitrophenols, 3,5-Dinitrosalicylic acid, 2,4-Dinitrobenzoic acid, 5-Sulfosalicylic acid, 2,5-Dihydroxy-1,4-benzene disulfonic acid, 3,5-Dinitrobenzoic acid, 8-Hydroxyquinoline-5-sulfonic acid, 4-Nitrophenol, 3,5-Dinitrosalicylaldehyde, 3,5-Dinitroaniline, Paratoluene sulfonic acid, 2-Mesitylene sulfonic acid, 2-(Trifluoromethyl)benzoic acid, 3,5-Dinitrobenzonitrile, and 2,4-Dinitrobenzene sulfonic acid, 3,5-Dinitrobenzoic acid, 2,4-Dinitrobenzoic acid, 2,4-Dinitrobenzene sulfonic acid, 2,6-Dinitrobenzene sulfonic acid, 3,5-Dinitrobenzene sulfonic acid, or 2,4-Dinitrophenol. Examples of fixatives are described in U.S. Pat. No. 5,422,277, issued Jun. 6, 1995, which is herein incorporated by reference.

A fixative can be used with a metal salt. The ratio of the fixative to the metal salt can be about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0. The ratio of the fixative to the metal salt can be at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0. In one embodiment, the ratio of formaldehyde to dichromate is about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0. In one embodiment, the ratio of formaldehyde to dichromate is at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0.

A concentration of a fixative in a composition of the provided invention can be about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1%. A concentration of a fixative in the compositions of the provided invention can be at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, or at least 1%.

In one embodiment, a stabilization composition is provided containing at least one fixative.

H. Inorganic Salts

The compositions of the provided invention can include one or more inorganic salts. Suitable inorganic salts for use in the compositions, methods, and kits of the provided invention can include, for example, NaCl, KCl, CaCl₂, ZnCl₂, NiCl₂, MgCl₂, or MnCl₂.

In one embodiment, a stabilization composition is provided comprising at least one inorganic salt.

I. Other Additives

The compositions of the provided invention can include one or more of PEG-200, PEG-300, PEG-400, PEG-600, PEG-1000, PEG-1450, PEG-3350, PEG-4000, PEG-6000, PEG-8000, PEG-20,000, imidazolidinyl urea, diazolidinyl urea, calcium propionate, sodium nitrate, sodium nitrite, sulfites, sulfur dioxide, sodium bisulfite, potassium hydrogen sulfite, disodium EDTA, ethanol, methylchloroisothiazolinone, DNase inhibitors, RNase inhibitors, and RNase.

The compositions of the provided invention can include protease inhibitors, e.g., PMSF Phenylmethyl sulfonyl fluoride, AEBSF-HCl, Amastatin-HCl, (epsilon)-Aminocaproic acid, (alpha)1-Antichymotypsin from human plasma, Antipain-HCL, Antithrombin III from human plasma, (alpha)1-Antitrypsin from human plasma ((alpha)1-proteinase inhibitor), APMSF-HCl (4-Amidinophenyl-methane sulfonyl-fluoride), Aprotinin (Trypsin inhibitor from bovine lung), Arphamenine A, Arphamenine B, Benzamidine-HCl, Bestatin-HCl, CA-074, CA-074-Me, Calpain Inhibitor I, Calpain Inhibitor II, Cathepsin Inhibitor Z-Phe-Gly-NHO-Bz-pMe, Chymostatin, DFP (Diisopropylfluoro-phosphate), Dipeptidylpeptidase IV Inhibitor H-Glu-(NHO-Bz)Pyr, Diprotin A, E-64, E-64d (EST), Ebelactone A, Ebelactone B, EDTA-Na_{z}, Elastatinal, Hirudin, Leuhistin, Leupeptin-Hemisulfate, (alpha)2-Macroglobulin from human plasma, PEFABLOC® SC (4-(2-Aminoethyl)-benzenesulfonyl fluoride hydrochloride), Pepstatin A, Phebestin, Phosphoramidon, TLCK(1-Chloro-3-tosylamido-7-amino-2-heptanone HCl), TPCK (1-Chloro-3-tosylamido-4-phenyl-2-butanone), Trypsin inhibitor from egg white (Ovomucoid), and Trypsin inhibitor from soybean.

The compositions of the provided invention can include phosphatase inhibitors including, for example, (−)-p-Bromotetramisole oxalate, Cantharidin, Microcystin LR from Microcystis aeruginosa, imidazole, sodium fluoride, sodium molybdate, sodium orthovanadate, sodium tartrate dihydrate, sodium pyrophosphate decahydrate, beta-glycerophosphate, and calyculin A from Discodermia calyx.

J. Cells in the Composition

The stabilization composition can comprise one or more isolated cells, or mixtures of different types of cells such as occur in blood samples, including isolated fetal cells, circulating tumor cells, white blood cells, or stem cells. In one embodiment, a method for stabilizing a cell or cellular component is provided comprising contacting a cell or cellular component (e.g., cell-free DNA) with a stabilization composition. In another embodiment, a method for stabilizing a fetal cell, circulating tumor cell, white blood cell, or stem cell is provided comprising contacting said fetal cell, circulating tumor cell, white blood cell, or stem cell with a stabilization composition. In another embodiment, a method for stabilizing a fetal cell, circulating tumor cell, white blood cell, or stem cell is provided comprising contacting said fetal cell, circulating tumor cell, white blood cell, or stem cell from a maternal blood sample with a stabilization composition. In another embodiment, a method for stabilizing a maternal cell in a maternal blood sample comprising cell-free DNA is provided comprising contacting the maternal cell with a stabilization composition of the provided invention.

K. Stabilization Composition Forms

The composition can be a solution. A solution can be added to another composition, e.g., a blood sample, resulting in dilution of the components of the stabilization solution. A stabilization solution can be provided with components that are at least 1.5× (×=“times”), 2×, 3×, 4×, 5×, 6×, 7×, 8×, 9×, 10×, 20×, 30×, 40×, 50×, 60×, 70×, 80×, 90×, or 100× the final concentration of the components when mixed with a sample, e.g., a blood sample. A stabilization solution can be diluted at least 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, or 1.5-fold when mixed with a sample, e.g., a blood sample or maternal blood sample.

In one embodiment, an at least 2×, 5×, or 10× stabilization composition is diluted with a maternal blood sample to provide a final 1× concentration of stabilization composition components. In another embodiment, an at least 2×, 5×, or 10× stabilization composition is diluted with a blood sample to provide a final 1× concentration of stabilization composition components.

I. Containers with Stabilization Compositions

The stabilization compositions can be provided in containers including tubes or syringes for drawing blood. Concentrated stabilization compositions can be provided in containers including tubes or syringes for drawing blood. Blood drawing tubes can include, for example, BD Vacutainer® PST™ Tubes with spray-coated lithium heparin and a polymer gel for plasma separation, BD Vacutainer® PST™ Tubes with spray-coated silica, BD Vacutainer® Heparin Tubes spray-coated with either lithium heparin or sodium heparin, BD Vacutainer® EDTA tubes, BD Vacutainer® Tubes with Acid Citric Dextrose (ACD), BD Microtainer® Blood Collection Tubes with lithium heparin/PST™ Gel, BD Microtainer® Blood Collection Tubes with lithium heparin, BD Microtainer® Plastic Clad Micro-Hematocrit Tubes with ammonium heparin.

The stabilization compositions can be provided in other containers, including a Rare-Cell™ blood collection tube (BCT) from Streck Innovations, cell-free DNA™ BCT from Streck Innovations, or Cyto-Chex® BCT from Streck Innovations.

In one embodiment, a BD Vacutainer® Heparin Tube is provided comprising a stabilization composition of the provided invention. In another embodiment, a BD Vacutainer® Tube with Acid Citric Dextrose (ACD) comprising a stabilization composition of the provided invention is provided. The stabilization composition in a container can be concentrated at least 2×, at least 5×, or at least 10× the final concentration when diluted with a sample, e.g., a blood sample.

H. Kits Containing Stabilization Compositions

Kits can be generated containing the compositions of the provided invention. A blood drawing tube, syringe, or other container can be included in a kit for obtaining and shipping blood samples. Other components of such kits can include written instructions. The written instructions can be for drawing blood, shipping a blood sample, or both. The kits can contain needles. The kits can contain labels that contain shipping information, e.g., address information for returning a kit to a kit provider. The kits can contain labels comprising information regarding the sample and/or the subject, and the labels can be placed on a container used for a blood draw to identify the container. The kits can be sent to a healthcare provider, e.g., a doctor, nurse, phlebotomist, surgeon, obstetrician/gynecologist, or pediatrician. Computer and internet based communications can be used in sending, tracking, or receiving a kit with a stabilization composition of the provided invention. Information related to a kit (e.g. type of tube/container sent, type of composition in a tube/container, type of sample, information on the subject from whom a sample is taken (e.g., age of the subject, duration of pregnancy, medical history) can be returned with a sample in a kit to the kit provider. This information can be input into a computer.

In one embodiment, a kit is provided comprising a tube comprising heparin and a stabilization composition of the provided invention. In another embodiment, a kit is provided comprising a tube comprising heparin and a stabilization composition of the provided invention comprising at least three other anticoagulants and two or more antioxidants. In another embodiment, a kit is provided comprising a tube comprising heparin and a stabilization composition of the provided invention comprising two or more antioxidants and one or more cross-linking agents. In another embodiment, a kit is provided comprising a tube comprising heparin and a stabilization composition of the provided invention including glycine, N-acetyl-L-cysteine (NAC), glutamine, and D-Mannitol, and optionally one or more anticoagulants, cell membrane stabilizers, or energy sources. The composition can lack formaldehyde or an agent that slows cell metabolism. The stabilization composition can be provided in a kit at at least 2.5×, at least 5×, or at least 10× the final concentration of the components of the stabilization composition when mixed with a sample.

In another embodiment, a kit comprising a tube comprising heparin and a stabilization composition of the provided invention is provided to a healthcare provider.

IV. Samples that can be Used with the Stabilization Composition

A. Maternal and Fetal Samples

The composition, methods, and kits of the provided invention can include use of maternal samples. Samples can be obtained from any animal in need of a diagnosis or prognosis or from an animal pregnant with a fetus in need of a diagnosis or prognosis. In one embodiment, a sample can be obtained from an animal suspected of being pregnant, pregnant, or that has been pregnant to detect the presence of a fetus or fetal abnormality. An animal of the present invention can be a human or a domesticated animal such as a cow, chicken, pig, horse, rabbit, dogs, cat, or goat. Samples derived from an animal, e.g., a human, can include, e.g., whole blood, plasma, serum, sweat, tears, peritoneal fluid, ear flow, sputum, lymph, bone marrow suspension, lymph, urine, saliva, semen, vaginal flow, fecal matter, cerebrospinal fluid, brain fluid, ascites, breast fluid, milk, secretions of the respiratory, intestinal or genitourinary tracts fluid, amniotic fluid (via, e.g., amniocentesis), a biopsy of the placenta (by, e.g., chorionic villi sampling, CVS), an umbilical cord blood sample, or a cervical swab.

In one embodiment, the sample is a maternal blood sample. Blood can be collected using any standard technique for blood-drawing including venipuncture. For example, blood can be drawn from a vein from the inside of the elbow or the back of the hand. To obtain a blood sample, a device known in the art can be used, e.g., a syringe or other vacuum suction device. In another embodiment, any blood drawing technique, method, protocol, or equipment that reduce the amount of cell lysis can be used, including but not limited to a large boar needle, a shorter length needle, a needle coating that increases laminar flow, e.g., teflon, a modification of the bevel of the needle to increase laminar flow, or techniques that reduce the rate of blood flow.

A maternal sample can contain one or more different types of fetal cells. A fetal cell can be any cell derived from a zygote, blastocyst, or embryo. A fetal cell can include, for example, a T cell, a B cell, a natural-killer (NK) cell, an antigen-presenting cell, an erythroblast, a nucleated erythrocyte (red blood cell), an enucleated red blood cell, a leukocyte, a pregnancy-associated progenitor cell (PAPCs), a fetal mesenchymal stem cell, a CD34+ cell (hematopoietic stem cell; HSC); a CD34+CD38+ cell, an epithelial cell, an endometrial cell, and a placental cell. A placental cell can include a trophoblast, e.g., syncytiotrophoblast (cell of the outer syncytial layer of the trophoblast) and a cytotrophoblast (cell of the inner layer of the trophoblast).

In one embodiment, fetal cells are isolated from maternal peripheral blood.

The sample can be an embryonic tissue, an embryo, a two-celled embryo, a four-celled embryo, an eight celled embryo, a 16-celled embryo, a 32-celled embryo, a 64-celled embryo, a 128-celled embryo, a 256-celled embryo, a 512-celled embryo, or a 1024-celled embryo.

Blood samples can be collected from a pregnant female at any time during fetal gestation. For example, blood samples can be collected from human females at 1-4, 4-8, 8-12, 12-16, 16-20, 20-24, 24-28, 28-32, 32-36, 36-40, 40-44, 48-52, or more than 52 weeks of fetal gestation. A blood sample can be obtained from a pregnant animal or human within 40, 36, 24, 22, 20, 18, 16, 14, 12, 10, 8, 6 or 4 weeks of conception or after a pregnancy has terminated. The sample can be taken during the first trimester (about the first three months of pregnancy), the 2^{nd} trimester (about months 4-6 of pregnancy), or the third trimester (about months 7-9 of pregnancy).

When obtaining a sample from an animal (e.g., blood sample), the amount of sample can vary. The amount of sample can vary depending upon animal size, its gestation period, and the condition being screened. In one embodiment, up to 50, 40, 30, 20, 10, 9, 8, 7, 6, 5, 4, 3, 2, or 1 mL of a sample is obtained. In another embodiment, 1-50, 2-40, 3-30, or 4-20 mL of sample is obtained. In another embodiment, more than 1, 5, 10, 15, 20, 25, 30, 35, 40, 45, 50, 55, 60, 65, 70, 75, 80, 85, 90, 95 or 100 mL of a sample is obtained. In another embodiment, between about 10-20 mL of a peripheral blood sample is obtained from a pregnant female.

The blood sample can be centrifuged to separate the plasma from the maternal cells. The plasma and maternal cell fractions are transferred to separate tubes and re-centrifuged. The plasma fraction contains cell-free fetal DNA and maternal DNA. Any standard DNA isolation technique can be used to isolate the fetal DNA and the maternal DNA including but not limited to QIAamp DNA Blood Midi Kit supplied by QIAGEN (Catalog number 51183).

The sample can be a serum sample. Fibrinogen and other clotting factors can be removed from the sample. In one embodiment, a method of stabilizing a fetal cell in a maternal blood sample is provided comprising contacting said cell with a stabilization composition of the provided invention. In another embodiment, a method of stabilizing a maternal cell in a maternal blood sample is provided comprising contacting said maternal cell with a stabilization composition of the provided invention. In another embodiment, a method of stabilizing a maternal cell in a maternal blood sample comprising cell-free DNA is provided comprising contacting said maternal cell with a stabilization composition of the provided invention.

B. Nucleic Acid Samples

The compositions of the provided invention can be added to solutions that comprise nucleic acids and cells. A nucleic acid can be any nucleic acid, e.g., genomic, plasmid, cosmid, yeast artificial chromosomes, RNA, mRNA, cell-free RNA or DNA, artificial or man-made DNA, including unique DNA sequences, and also DNA that has been reverse transcribed from an RNA sample, such as cDNA. The sequence of RNA can be determined according to the invention, e.g., if it is capable of being made into a double stranded DNA form to be used as template DNA.

In one embodiment, a stabilization composition of the provided invention is added to a sample comprising fetal nucleic acids. In another embodiment, a stabilization composition of the provided invention is added to a sample including fetal and maternal nucleic acids. In another embodiment, the sample is a maternal blood sample including cell-free fetal and maternal nucleic acids. Nucleic acid can include fetal DNA, fetal RNA, maternal DNA, or maternal RNA. In one embodiment, the sample is a maternal blood sample that includes fetal and maternal nucleic acids and fetal and maternal cells.

C. Tumor Cells

Cells that can be stabilized by the compositions, methods, and kits of the provided invention include circulating tumor cells (CTCs). CTC's include those cancer cells which have become detached from the primary tumor, or disseminated and micrometastasized cancer cells. Because the spread of these cells is usually connected with the vascularization of the primary tumor, CTCs can be found in particular in the blood, with bone marrow and lymph nodes also being suitable sources for samples.

A rare cell subtype can include any type of cell classification based on a phenotype, a genotype of the cell, or any combination thereof, including, but not limited to, circulating cancer stem cells, circulating cancer non-stem cells, tumorigenic cells, non-tumorigenic cells, apoptotic cells, non-apoptotic cells, terminal cells, non-terminal cells, proliferative cells, non-proliferative cells, cells derived from specific tissues, cells derived from specific cancer tissues, disseminated cancer cells, micrometastasized cancer cells, or cells associated with a condition. Other examples of subtypes of rare cells include those of specific tissue of origin such as circulating endothelial cells or circulating lung, liver, breast or prostate cancer cells. Other cell classifications and cell subtypes can include cells with specific cancer phenotypes. For example, breast cancer cells can have at least 6 different phenotypes, such as luminal/epithelial, basal/myoepithelial, mesenchymal, ErbB2, hormonal, and hereditary. Phenotypes of a cancer cell are discussed in US Patent Application Publication No. 2004/0191783.

In one embodiment, a method of stabilizing a circulating tumor cell is provided comprising contacting said circulating tumor cell with a stabilization composition of the provided invention.

D. Stem Cells

Cells that can be stabilized by the compositions, methods, and kits of the provided invention include stem cells. There are several qualities of stem cells. Stem cells are capable of dividing to produce daughter cells. They can exhibit self-maintenance or renewal over the lifetime of the organism. Stem cells are capable of reproducing by dividing symmetrically or asymmetrically to produce new stem cells. Symmetric division occurs when one stem cell divides into two daughter stem cells. Asymmetric division occurs when one stem cell forms one new stem cell and one progenitor cell. Symmetric division is a source of renewal of stem cells. This permits stem cells to maintain a consistent level of stem cells in an embryo or adult mammal. Stem cells can generate large number of progeny. Stem cells may produce a large number of progeny through the transient amplification of a population of progenitor cells. Stem cells can retain their multilineage potential over time. Stem cells are a source of differentiated tissue cells, so they retain their ability to produce multiple types of progenitor cells, which will in turn develop into specialized tissue cells. Stem cells can generate new cells in response to injury or disease. This is essential in tissues which have a high turnover rate or which are more likely to be subject to injury or disease, such as the epithelium of blood cells.

Stem cells can be distinguished depending on their different ability to differentiate into different kinds of tissues (different degree of “potency”). Stem cells are distributed in all tissues, and are available from sources like bone marrow, dental pulp, adipose tissue, peripheral blood, umbilical cord and fetal membrane.

Adult stem cells, for example, mesenchymal stem cells (MSCs), are adherent, multipotent stem cells that express a panel of surface antigens. Human MSCs can be found in bone marrow, amniotic membrane, chorial membrane, Wharton gel, cord blood and placenta, dental pulp, and lipoaspirates.

Adult stem cells can be derived from adipose.

In one embodiment, a method of stabilizing a stem cell is provided comprising contacting said stem cell with a stabilization composition of the provided invention.

E. White Blood Cells

The compositions, methods, and kits of the provided invention can be used to stabilize white blood cells (WBCs), or leukocytes. Leukocytes are derived from multipotent hematopoietic stem cells in the bone marrow. Leukocytes are found throughout the body, including the blood and lymphatic system. White blood cells include granulocytes or agranulocytes. Granulocytes include neutrophils, basophils, and eosinophiles. Agranulocytes include lymphocytes, monocytes, and macrophages. Lymphocytes include T-cells, B-cells, and natural killer cells. T cells include CD4+ (helper) T-cells, CD8+ (cytotoxic) T-cells, and γδ (gammadelta) T cells. B cells include plasma B cells, memory B cells, B-1 cells, B-2 cells, marginal-zone B-cells, and follicular B cells. Monocytes include classical monocytes and non-classical monocytes.

Stabilized white blood cells can be used to study immune diseases and to generate expression data.

In one embodiment, a method of stabilizing a white blood cell is provided comprising contacting said white blood cell with a stabilization composition of the provided invention.

### V. Enrichment/Purification

The stabilization compositions of the provided invention can be used in methods for enriching, concentrating, or purifying cells, e.g., fetal cells, circulating tumor cells, white blood cells, or stem cells.

A. Concentration

A maternal sample can be enriched for one or more fetal cells or fetal nucleic acid using one or more any methods known in the art (e.g. Guetta, E M et al. Stem Cells Dev, 13(1):93-9 (2004), which is herein incorporated by reference in its entirety) or described herein. The enrichment increases the concentration of one or more rare cells or the ratio of one or more rare cells to non-rare cells in the sample. For example, enrichment can increase the concentration of an analyte of interest such as a fetal cell by a factor of at least 2, 4, 6, 8, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, 1,000,000,000, 2,000,000,000, or 5,000,000,000 fold over its concentration in the original sample. In particular, when enriching one or more fetal cells from a maternal peripheral venous blood sample, the initial concentration of the one or more fetal cells in a sample can be about 1:50,000,000 and it can be increased to at least 1:5,000 or 1:500. Rare cells can also be enriched in a sample by the removal of fluid. A fluid sample (e.g., a blood sample) of greater than 10, 15, 20, 50, or 100 mL total volume can comprise rare components of interest, and it can be concentrated such that the rare component of interest is concentrated into a concentrated solution of less than 0.5, 1, 2, 3, 5, or 10 mL total volume.

The stabilization compositions of the provided invention can be used in methods for concentrating cells, e.g., fetal cells, circulating tumor cells, white blood cells, or stem cells. In one embodiment, a method of concentrating a fetal cell, circulating tumor cell, white blood cell, or stem cell is provided comprising contacting said fetal cell, circulating tumor cell, white blood cell, or stem cell with a stabilization composition of the provided invention and concentrating said fetal cell, circulating tumor cell, white blood cell, or stem cell by density gradient centrifugation, size-based separation, affinity-based enrichment, or red-blood cell lysis.

B. Density Gradient Centrifugation

Density gradient centrifugation can be used in the methods described herein to enrich cells stabilized using the compositions herein. Density gradient centrifugation is a method of separating cells based on the different densities of cell types in a mixture. The method can be used in a single step to separate cells into two compartments which contain cells that are either lighter or heavier than a specific density of the gradient material used. Density gradient centrifugation can be carried out through repetitive steps based on a series of different density gradients or in combination with affinity separation, cell panning, cell sorting, and the like. Alternatively, density gradient centrifugation can be performed using multiple layers of the different gradient densities. This method allows cells of different densities to form zones or bands at their corresponding densities after centrifugation. The cells in the different zones are then collected by placing a pipette at the appropriate location. Methods for enriching specific cell-types by density gradient centrifugation are described in U.S. Pat. No. 5,840,502, which is herein incorporated by reference in its entirety.

U.S. Pat. No. 5,432,054 describes a technique for separation of fetal nucleated red blood cells using a tube having a wide top and a narrow, capillary bottom made of polyethylene. Centrifugation using a variable speed program results in a stacking of red blood cells in the capillary based on the density of the molecules. The density fraction containing low-density red blood cells, including fetal red blood cells, is recovered and then differentially hemolyzed to preferentially destroy maternal red blood cells. A density gradient in a hypertonic medium is used to separate red blood cells, now enriched in the fetal red blood cells from lymphocytes and ruptured maternal cells. The use of a hypertonic solution shrinks the red blood cells, which increases their density, and facilitates purification from the more dense lymphocytes. After the fetal cells have been isolated, fetal DNA can be purified using standard techniques in the art.

The density gradient medium can be colloidal polyvinylpyrrolidone-coated silica (e.g. PercolD, Nycodenz), a nonionic polysucrose (Ficoll) either alone or with sodium diatrizoate (e.g. Ficoll-Paque or Histopaque), or mixtures thereof. The density of the reagent employed is selected to separate the fetal cells of interest from other blood components.

In one embodiment, a method of enriching a fetal cell from a maternal blood sample is provided comprising contacting said fetal cell with a stabilization composition of the provided invention and enriching said fetal cell by density gradient centrifugation. In one embodiment, the fetal cell is a fetal nucleated red blood cell. In another embodiment, a method of enriching a white blood cell is provided comprising contacting said white blood cell with a stabilization composition of the provided invention and enriching said cell by density gradient centrifugation.

Enrichment can occur using one or more types of separation modules. Several different modules are described herein, all of which can be fluidly coupled with one another in series for enhanced performance.

C. Enrichment by Lysis

In one embodiment, enrichment occurs by selective cell lysis. In one embodiment red blood cells are obtained from a maternal blood sample that has been treated with a stabilization composition of the provided invention. The cells can be obtained from the treated maternal blood sample for example by centrifugation. The cells are then treated with a selective red blood cell lysis agent that preferentially lyses the maternal enucleated red blood cells as compared to the nucleated fetal red blood cells.

Suitable selective red blood cell lysis compositions include, for example, Erythrolyse Red Blood Cell Lysing Buffer from AbD Serotec (Catalog No. BUF04B), RBC Lysis Solution from AppliChem GmbH (Catalog No. A4617), BD FACS Lysing Solution from BD Biosciences (Catalog No. 349202), EasyLyse™ Erythrocyte-Lysing Reagent from Dako (Catalog No. 236430), Uti-Lyse™, Erythrocyte-Lysing Reagent for Dako (Catalog No. S332530), Human Erythrocyte Lysing Kit from R&D Systems (Catalog No. WL1000), Fixative-Free Red Cell Lysing Solution for Flow Cytometric Applications from Invitrogen (Product code: HYL-250), RBC Lysis Solution from 5 PRIME (Catalog No. 2301300), VersaLyse Lysing Solution from Beckman Coulter (Catalog No. A09777), FCM Lysing Solution (1×) from Santa Cruz Biotechnology, Inc. (Catalog No. sc-3621), EasySep RBC Lysis Buffer from StemCell Technologies, Inc. (Catalog No. 20110), Red Blood Cell Lysing Buffer Hybr-Max® from Sigma-Aldrich (Catalog No. R7757), RBC Lysis Buffer (10×) from BioLegend (Catalog No. 420301). Other RBC lysing agents include Amyloid β-peptide (Aβ)25-35 (Mattson M P et al. (1997) Brain Research 771:147-153).

U.S. Pat. No. 6,869,798 describes reagents for red blood cell lysis. U.S. Pat. No. 4,617,275 (to Matsuda, et al.) describes the use of a lysing reagent comprising quaternary ammonium salts to provide adequate red cell lysis without excessively damaging the white blood cells for the purpose of electrical impedance measurement of at least three subpopulations of leukocytes. The lysing reagent contains citric acid to assist in removal of the interfering red cell ghosts. The analysis method requires the use of a diluent solution as a co-reagent. The diluent contains a buffer comprised of boric acid and sodium borate.

U.S. Pat. No. 4,637,986 (to Brown, et al.) describes a flow cytometry lysing reagent for producing a 3-part differential of leukocytes. The lysing reagent is a hypotonic aqueous solution enabling hypotonic lysis of red blood cells. The lysing reagent comprises a leukoprotective agent for preserving the lymphocyte cellular integrity during analysis, and buffers to provide the correct pH environment for optimal lysis.

In one embodiment, a blood sample can be combined with an agent that selectively lyses one or more cells or components in a blood sample. In one embodiment, platelets and/or enucleated red blood cells are selectively lysed to generate a sample enriched in nucleated cells, such as fetal nucleated red blood cells (fnRBC's), maternal nucleated blood cells (mnBC), or epithelial cells. fnRBCs can be subsequently separated from mnBC's using, e.g., size-based separation, antibodies, antigen-i affinity or differences in hemoglobin. In one embodiment, one or more fetal cells can be selectively lysed and their nuclei released when a blood sample including one or more fetal cells is combined with deionized water. Such selective lysis allows for the subsequent enrichment of fetal nuclei using, e.g., size or affinity based separation.

D. Size-Based Enrichment

In one embodiment, enrichment of rare cells occurs using one or more size-based separation modules. Examples of size-based separation modules include filtration modules, sieves, matrixes, etc. Examples of size-based separation modules contemplated for use in the methods of the provided invention include those disclosed in International Publication No. WO 2004/113877, which is herein incorporated by reference in its entirety. Other size based separation modules are disclosed in International Publication No. WO 2004/0144651 and US. Patent Application Publication Nos. US20080138809A1 and US20080220422A1, which are herein incorporated by reference in their entirety.

In one embodiment, a size-based separation module comprises one or more arrays of obstacles forming a network of gaps. The obstacles are configured to direct particles as they flow through the array/network of gaps into different directions or outlets based on the particle's hydrodynamic size. For example, as a blood sample flows through an array of obstacles, nucleated cells or cells having a hydrodynamic size larger than a predetermined size, e.g., 8 microns, are directed to a first outlet located on the opposite side of the array of obstacles from the fluid flow inlet, while the enucleated cells or cells having a hydrodynamic size smaller than a predetermined size, e.g., 8 microns, are directed to a second outlet also located on the opposite side of the array of obstacles from the fluid flow inlet.

An array can be configured to separate cells smaller or larger than a predetermined size by adjusting the size of the gaps, obstacles, and offset in the period between each successive row of obstacles. For example, in one embodiment, obstacles or gaps between obstacles can be up to 10, 20, 50, 70, 100, 120, 150, 170, or 200 microns in length or about 2, 4, 6, 8 or 10 microns in length. In one embodiment, an array for size-based separation includes more than 100, 500, 1,000, 5,000, 10,000, 50,000 or 100,000 obstacles that are arranged into more than 10, 20, 50, 100, 200, 500, or 1000 rows. In one embodiment, obstacles in a first row of obstacles are offset from a previous (upstream) row of obstacles by up to 50% the period of the previous row of obstacles. In one embodiment, obstacles in a first row of obstacles are offset from a previous row of obstacles by up to 45, 40, 35, 30, 25, 20, 15 or 10% the period of the previous row of obstacles. Furthermore, the distance between a first row of obstacles and a second row of obstacles can be up to 10, 20, 50, 70, 100, 120, 150, 170 or 200 microns. A particular offset can be continuous (repeating for multiple rows) or non-continuous. In one embodiment, a separation module includes multiple discrete arrays of obstacles fluidly coupled such that they are in series with one another. Each array of obstacles has a continuous offset. But each subsequent (downstream) array of obstacles has an offset that is different from the previous (upstream) offset. In one embodiment, each subsequent array of obstacles has a smaller offset that the previous array of obstacles. This arrangement allows for a refinement in the separation process as cells migrate through the array of obstacles. Thus, a plurality of arrays can be fluidly coupled in series or in parallel, (e.g., more than 2, 4, 6, 8, 10, 20, 30, 40, 50). Fluidly coupling separation modules (e.g., arrays) in parallel allows for high-throughput analysis of the sample, such that at least 1, 2, 5, 10, 20, 50, 100, 200, or 500 mL per hour flows through the enrichment modules or at least 1, 5, 10, or 50 million cells per hour are sorted or flow through the device.

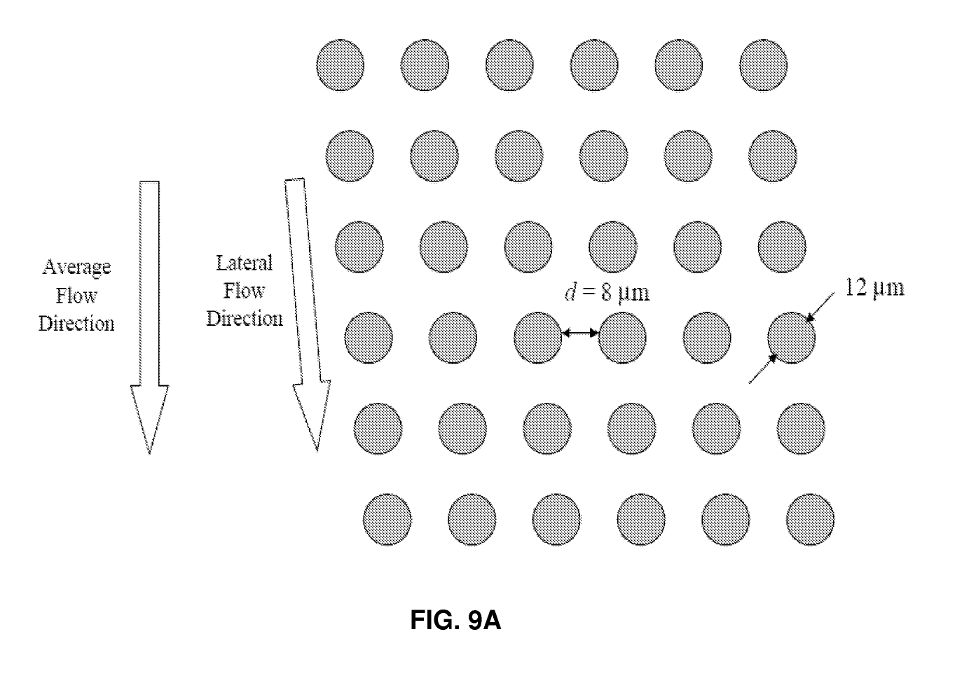

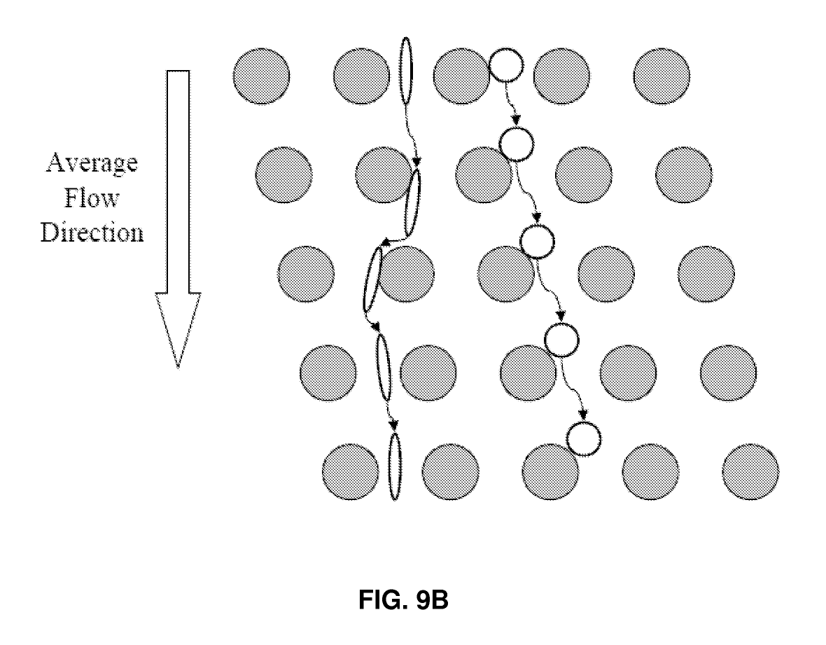

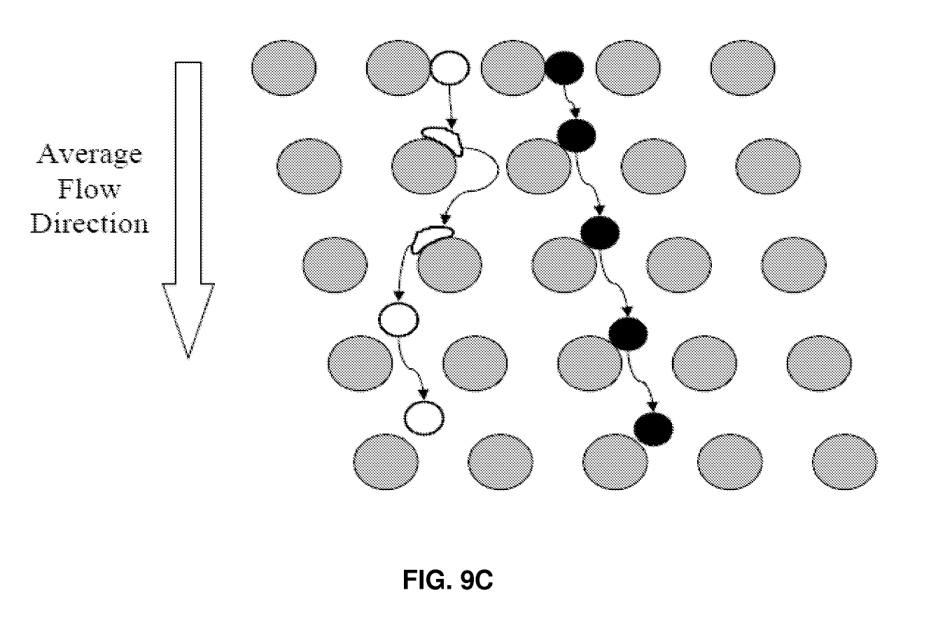

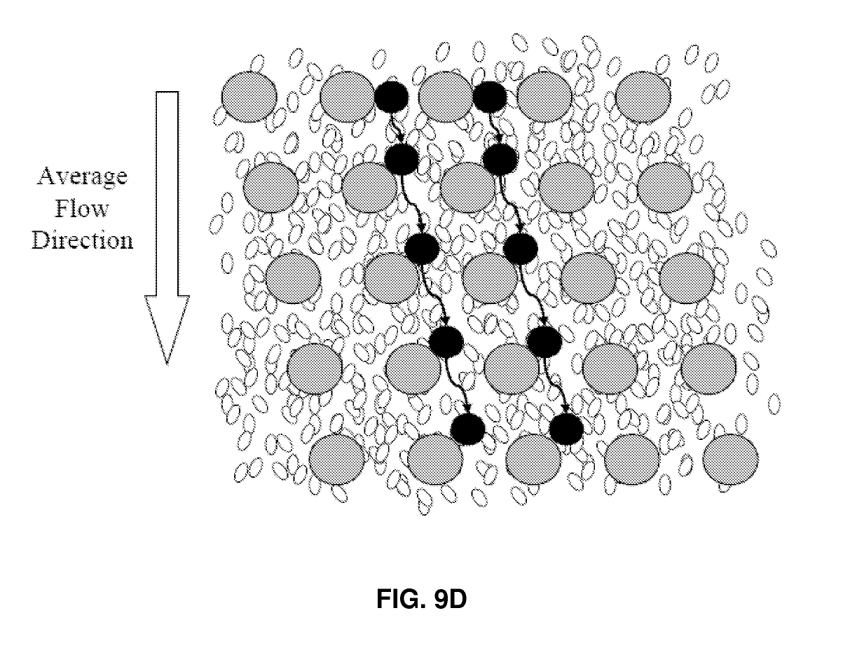

FIG. 9A illustrates an example of a size-based separation module. In one embodiment, obstacles (which can be of any shape) are coupled to a flat substrate to form an array of gaps. A transparent cover or lid can be used to cover the array. The obstacles form a two-dimensional array with each successive row shifted horizontally with respect to the previous row of obstacles, where the array of obstacles directs one or more components having a hydrodynamic size smaller than a predetermined size in a first direction and one or more components having a hydrodynamic size larger that a predetermined size in a second direction. For enriching epithelial cells from enucleated cells, the predetermined size of gaps in an array of obstacles can be 6-12 μm or 6-8 μm. For enriching one or more fetal cells from a mixed sample (e.g., maternal blood sample) the predetermined size of gaps in an array of obstacles can be between 4-10 μm or 6-8 μm. The flow of sample into the array of obstacles can be aligned at a small angle (flow angle) with respect to a line-of-sight of the array. Optionally, the array is coupled to an infusion pump to perfuse the sample through the obstacles. The flow conditions of the size-based separation module described herein are such that cells are sorted by the array with minimal damage. This allows for downstream analysis of intact cells and intact nuclei to be more efficient and reliable.

In one embodiment, a size-based separation module comprises an array of obstacles configured to direct cells larger than a predetermined size to migrate along a line-of-sight within the array (e.g., towards a first outlet or bypass channel leading to a first outlet), while directing cells and analytes smaller than a predetermined size to migrate through the array of obstacles in a different direction than the larger cells (e.g., towards a second outlet). Such embodiments are illustrated in part in FIG. 9B-9D.

A variety of enrichment protocols can be utilized. In one embodiment the cells are handled gently to reduce mechanical damage to the cells or their DNA. This gentle handling can serve to preserve the small number of one or more fetal cells in the sample. Integrity of the nucleic acid being evaluated is an important feature to permit the distinction between the genomic material from the one or more fetal cells and other cells in the sample. In particular, the enrichment and separation of one or more fetal cells using the arrays of obstacles provides gentle treatment which minimizes cellular damage. Moreover, this gentle treatment maximizes nucleic acid integrity, permits exceptional levels of separation, and allows for the ability to subsequently utilize various formats to analyze the genome of the cells.

In one embodiment, a method of enriching a fetal cell from a maternal blood sample is provided comprising contacting said fetal cell with a stabilization composition of the provided invention and enriching said fetal cell using size-based separation. The size-based separation can comprise a two-dimensional array of staggered obstacles. The fetal cell can be a fetal nucleated red blood cell.

E. Affinity-Based Enrichment

In one embodiment, enrichment of one or more rare cells (e.g., one or more fetal cells or circulating tumor cells) occurs using one or more capture modules that selectively inhibit the mobility of one or more cells of interest. In one embodiment, a capture module is fluidly coupled downstream to a size-based separation module. Capture modules can include a substrate having multiple obstacles that restrict the movement of cells or analytes greater than a predetermined size. Examples of capture modules that inhibit the migration of cells based on size are disclosed in U.S. Pat. No. 5,837,115 and 6,692,952, which are herein incorporated by reference in their entirety.

In one embodiment, a capture module includes a two dimensional array of obstacles that selectively filters or captures cells or analytes having a hydrodynamic size greater than a particular gap size (predetermined size), International Publication No. WO 2004/113877, which is herein incorporated by reference in its entirety.

In one embodiment a capture module captures analytes (e.g., cells of interest or not of interest) based on their affinity for a binding moiety. For example, an affinity-based separation module that can capture cells or analytes can include an array of obstacles adapted for permitting sample flow through, but for the fact that the obstacles are covered with binding moieties that selectively bind one or more analytes (e.g., cell populations) of interest (e.g., one or more red blood cells, fetal cells, epithelial cells or nucleated cells) or analytes not-of-interest (e.g., white blood cells). Arrays of obstacles adapted for separation by capture can include obstacles having one or more shapes and can be arranged in a uniform or non-uniform order. In one embodiment, a two-dimensional array of obstacles is staggered such that each subsequent row of obstacles is offset from the previous row of obstacles to increase the number of interactions between the analytes being sorted (separated) and the obstacles. Other types of binding modules can be used.

In one embodiment, a method of enriching a fetal cell is provided comprising contacting said fetal cell with a stabilization composition of the provided invention and enriching said cell using antibody-based enrichment. In another embodiment, the fetal cell is a fetal nucleated red blood cell.

1. Antibody Fragments

In one embodiment of the invention the binding member is a fragment of an antibody, e.g., an antigen binding fragment or a variable region. Examples of antibody fragments useful with the present invention include Fab, Fab′, F(ab′) 2 and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′) 2 fragment that has two antigen binding fragments which are capable of cross-linking antigen, and a residual other fragment (which is termed pFc′).

Additional fragments can include diabodies, linear antibodies, single-chain antibody molecules, and multispecific antibodies formed from antibody fragments.

The antibody fragments Fab, Fv and scFv differ from whole antibodies in that the antibody fragments carry only a single antigen-binding site. Recombinant fragments with two binding sites have been made in several ways, for example, by chemical cross-linking of cysteine residues introduced at the C-terminus of the VH of an Fv (Cumber et al., 1992 which is herein incorporated by reference in its entirety), or at the C-terminus of the VL of an scFv (Pack and Pluckthun, 1992, which is herein incorporated by reference in its entirety), or through the hinge cysteine residues of Fab's (Carter et al., 1992, which is herein incorporated by reference in its entirety).

Antibody fragments retain some or essentially all the ability of an antibody to selectively bind with its antigen or receptor. Examples of antibody fragments include the following:

Fab is the fragment that contains a monovalent antigen-binding fragment of an antibody molecule. A Fab fragment can be produced by digestion of whole antibody with the enzyme papain to yield an intact light chain and a portion of one heavy chain.

Fab′ is the fragment of an antibody molecule and can be obtained by treating whole antibody with pepsin, followed by reduction, to yield an intact light chain and a portion of the heavy chain. Two Fab′ fragments are obtained per antibody molecule. Fab1 fragments differ from Fab fragments by the addition of a few residues at the carboxyl terminus of the heavy chain CH 1 domain including one or more cysteines from the antibody hinge region.

(Fab′)2 is the fragment of an antibody that can be obtained by treating whole antibody with the enzyme pepsin without subsequent reduction. F(ab′)2 is a dimer of two Fab′ fragments held together by two disulfide bonds.

Fv is the minimum antibody fragment that contains a complete antigen recognition and binding site. This region consists of a dimer of one heavy and one light chain variable domain in a tight, non-covalent association (VH-V L dimer). It is in this configuration that the three CDRs of each variable domain interact to define an antigen binding site on the surface of the VH-V L dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The antibody can be a single chain antibody (“SCA”), defined as a genetically engineered molecule containing the variable region of the light chain, the variable region of the heavy chain, linked by a suitable polypeptide linker as a genetically fused single chain molecule. Such single chain anti-bodies are also referred to as “single-chain Fv” or “sFv” antibody fragments. Generally, the Fv polypeptide further comprises a polypeptide linker between the VH and VL domains that enables the sFv to form the desired structure for antigen binding.

The antibody fragments according to the invention can be produced in any suitable manner known to the person skilled in the art. Several microbial expression systems have already been developed for producing active antibody fragments, e.g., the production of Fab in various hosts, such as E. coli, yeast, and the filamentous fungus Trichoderma reesei are known in the art. The recombinant protein yields in these alternative systems can be relatively high (1-2 g/l for Fab secreted to the periplasmic space of E. coli in high cell density fermentation or at a lower level, e.g. about 0.1 mg/l for Fab in yeast in fermenters, and 150 mg/l for a fusion protein CBHI-Fab and 1 mg/l for Fab in Trichoderma in fermenters and such production is very cheap compared to whole antibody production in mammalian cells (hybridoma, myeloma, CHO).

The fragments can be produced as Fab's or as Fv's, but additionally it has been shown that a VH and a VL can be genetically linked in either order by a flexible polypeptide linker, which combination is known as an scFv.

2. Natural Single Domain Antibodies

Heavy-chain antibodies (HCAbs) are naturally produced by camelids (camels, dromedaries and llamas). HCAbs are homodimers of heavy chains only, devoid of light chains and the first constant domain (Hamers-Casterman et al., 1993, which is herein incorporated by reference in its entirety). The possibility to immunize these animals allows for the cloning, selection and production of an antigen binding unit consisting of a single-domain only. Furthermore these minimal-sized antigen binding fragments are well expressed in bacteria, interact with the antigen with high affinity and are very stable.

New or Nurse Shark Antigen Receptor (NAR) protein exists as a dimer of two heavy chains with no associated light chains. Each chain is composed of one variable (V) and five constant domains. The NAR proteins constitute a single immunoglobulin variable-like domain (Greenberg et al) which is much lighter than an antibody molecule.

3. Fetal Markers for Enrichment

Fetal cell markers (e.g., fetal proteins) can be used for enriching fetal cells. Proteins expressed from the genes hPL, CHS2, KISS1, GDF15, CRH, TFP12, CGB, LOC90625, FN1, COL1A2, PSG9, PSG1, AFP, APOC3, SERPINC1, AMBP, CPB2, ITIH1, APOH, HPX, beta-hCG, AHSG, APOB, or J42-4d can be used for fetal cell enrichment. In one embodiment, one or more antibodies that bind a protein expressed from the genes hPL, CHS2, KISS1, GDF15, CRH, TFP12, CGB, LOC90625, FN1, COL1A2, PSG9, PSG1, AFP, APOC3, SERPINC1, AMBP, CPB2, ITIH1, APOH, HPX, beta-hCG, AHSG, APOB, or J42-4d is used to enrich fetal cells. In one embodiment samples are enriched for one or more fetal nucleated RBCs by anti-CD71 or anti-GLA selection. In another embodiment one or more trophoblasts are enriched by anti-HLA-G or anti-EGFR selection.

In one embodiment, a method of enriching a fetal cell is provided comprising contacting said fetal cell with a stabilization composition of the provided invention and enriching said fetal cell using one or more antibodies that target one or more of the proteins hPL, CHS2, KISS1, GDF15, CRH, TFP12, CGB, LOC90625, FN1, COL1A2, PSG9, PSG1, AFP, APOC3, SERPINC1, AMBP, CPB2, ITIH1, APOH, HPX, beta-hCG, AHSG, APOB, or J42-4d. In one embodiment, a method of enriching a fetal nucleated red blood cell is provided comprising contacting said fetal nucleated red blood cell with a stabilization composition of the provided invention and enriching said fetal nucleated red blood cell using anti-CD71 or anti-GLA antibodies.

F. Dielectrophoretic Enrichment

In one embodiment an electric field exert forces on a neutral but polarisable particle, such as cell, suspended in a liquid. According to this particular electrokinetic principle, which is called dielectrophoresis (DEP), a neutral particle, when subject to non-uniform electric fields, experiences a net force directed towards locations with increasing (positive dielectrophoresis—pDEP) or decreasing (negative dielectrophoresis—nDEP) field intensities. More specifically, a particle can be subject to pDEP or nDEP according to the (frequency-dependent) electrical properties of the particle and its suspending medium, the particle dimension and the gradient of the electric field. In one embodiment, the electric field is generated by a silicon chip directly interfaced to a microchamber containing living or non-living particles in liquid suspension. The microchamber is confined between the chip surface and a conductive transparent lid spaced tens of microns apart. The chip surface implements a two dimensional array of microlocations, each consisting of a surface electrode, embedded sensors and logic. The electrodes induce suitable closed nDEP cages in the spatial region above selected microsites, within which single particles may be trapped and levitated individually. Step by step, DEP potential cages can be moved around the device plane concurrently and independently, thus grabbing and dragging single cells and/or microbeads to or from any microchamber location. Separation of heterogeneous populations can be performed by either exploiting DEP spectrum characterisation (i.e. using the frequency-dependent DEP force changing from positive to negative or vice versa) or by using labelling techniques based on functionalised microbeads or fluorescent dyes.

In another embodiment an apparatus can be used to enrich a particle such as a fetal cell by establishing closed dielectrophoretic potential cages and precise displacement thereof. The apparatus can comprise a first array of selectively addressable electrodes, lying on a substantially planar substrate and facing toward a second array comprising one electrode. The arrays define the upper and lower bounds of a micro-chamber where particles are placed in liquid suspension. By applying in-phase and counter-phase periodic signals to electrodes, one or more independent potential cages can be established which cause particles to be attracted to or repelled from cages according to signal frequency and the dielectric characteristics of the particles and suspending medium. By properly applying voltage signal patterns into arrays, cages may trap one or more particles, thus permitting them to levitate steadily and/or move. In one embodiment, an array can be integrated on a semiconductor substrate, displacement of particles can be monitored by embedded sensors.

G. Enrichment by Apoptosis

In one embodiment, enrichment involves detection and/or isolation of one or more rare cells or rare DNA (e.g., one or more fetal cells, circulating tumor cells, or fetal DNA) by selectively initiating apoptosis in the one or more rare cells. This enrichment can be accomplished, for example, by subjecting a sample that includes rare cells (e.g. a mixed sample) to hyperbaric pressure (increased levels of CO₂; e.g. 4% CO₂). This process will selectively initiate condensation and/or apoptosis in the one or more rare or fragile cells in the sample (e.g., one or more fetal cells). Once the one or more rare cells (e.g., one or more fetal cells) begin apoptosis, their nuclei will condense and optionally be ejected from the rare cells. At that point, the one or more rare cells or nuclei can be detected using any technique known in the art to detect condensed nuclei, including DNA gel electrophoresis, in situ labeling fluorescence labeling, and in situ labeling of DNA nicks using terminal deoxynucleotidyl transferase (TdT)-mediated dUTP in situ nick labeling (TUNEL) (Gavrieli, Y., et al. J. Cell Biol. 119:493-501 (1992), which is herein incorporated by reference in its entirety), and ligation of DNA strand breaks having one or two-base 3′ overhangs (Taq polymerase-based in situ ligation; Didenko V., et al. J. Cell Biol. 135:1369-76 (1996), which is herein incorporated by reference in its entirety).

In one embodiment ejected nuclei can further be detected using a size based separation module adapted to selectively enrich nuclei and other analytes smaller than a predetermined size (e.g. 6 microns) and isolate them from cells and analytes having a hydrodynamic diameter larger than 6 microns. Thus, in one embodiment, the present invention contemplated detecting one or more fetal cells/fetal DNA and optionally using such fetal DNA to diagnose or prognose a condition in a fetus. Such detection and diagnosis can occur by obtaining a blood sample from the female pregnant with the fetus, enriching the sample for cells and analytes larger than 8 microns using, for example, an array of obstacles adapted for size-base separation where the predetermined size of the separation is 8 microns (e.g. the gap between obstacles is up to 8 microns). Then, the enriched product is further enriched for red blood cells (RBC's) by oxidizing the sample to make the hemoglobin paramagnetic and flowing the sample through one or more magnetic regions. This selectively captures the RBC's and removes other cells (e.g. white blood cells) from the sample. Subsequently, the fnRBC's can be enriched from mnRBC's in the second enriched product by subjecting the second enriched product to hyperbaric or hypobaric pressure or other stimulus that selectively causes the one or more fetal cells to begin apoptosis and condense/eject their nuclei. Such condensed nuclei are then identified/isolated using, e.g., laser capture microdissection or a size based separation module that separates components smaller than 3, 4, 5 or 6 microns from a sample. Such fetal nuclei can then by analyzed using any method known in the art or described herein.

H. Flow Cytometry

Flow cytometry techniques can be used in the methods of the provided invention. Flow cytometry techniques can be used to enrich fetal cells (Herzenberg et al., PNAS 76: 1453-1455 (1979); Bianchi et al., PNAS 87: 3279-3283 (1990); Bruch et al., Prenatal Diagnosis 11: 787-798 (1991)). In one embodiment, one or more rare cells (e.g., one or more fnRBCs, placental cells, circulating tumor cells, etc.) can be enriched or purified using flow cytometry, fluorescent activated cell sorting (FACS) or microfluidic fluorescent cell sorting (e.g. the Cellula platform). In one embodiment one or more molecules (e.g., nucleic acids, proteins) in a rare cell of interest (e.g., fnRBC, placental cell, etc.) can be fluorescently labeled. For binding proteins, a fluorescent molecule can be attached a binding moiety, e.g., an antibody or antibody-based fragment. For enriching cells based on binding nucleic acids, a fluorescent label can be attached to a nucleic acid, e.g., a DNA or RNA probe. Techniques can include RNA-FISH. The probe can be a molecular beacon probe, in which the probe can anneal to form a hairpin that juxtaposes a fluorescent molecule attached to one end of the probe with a quenching moiety attached to the other end of the probe. In the hairpin formation, the probe is unable to fluoresce.

Gene products (e.g., transcripts or proteins) expressed from hPL, CHS2, KISS1, GDF15, CRH, TFP12, CGB, LOC90625, FN1, COL1A2, PSG9, PSG1, AFP, APOC3, SERPINC1, AMBP, CPB2, ITIH1, APOH, HPX, beta-hCG, AHSG, APOB, or J42-4d can be fluorescently labeled and used to enrich a fetal cell by flow cytometry.

In one embodiment, a method for enriching a fetal cell from a maternal blood sample is provided comprising contacting said fetal cell with a stabilization composition of the provided invention and enriching said fetal cell by flow cytometry. The fetal cell can be a fetal nucleated red blood cell. In another embodiment, a method of enriching a fetal cell from a maternal blood sample is provided comprising contacting said fetal cell with a stabilization composition of the provided invention and enriching said fetal cell by flow cytometry by fluorescently labeling one or more gene products expressed from the genes hPL, CHS2, KISS1, GDF15, CRH, TFP12, CGB, LOC90625, FN1, COL1A2, PSG9, PSG1, AFP, APOC3, SERPINC1, AMBP, CPB2, ITIH1, APOH, HPX, beta-hCG, AHSG, APOB, or J42-4d

I. Magnetic-Based Enrichment

In one embodiment, when the analyte desired to be separated (e.g., red blood cells or white blood cells) is not ferromagnetic or does not have a potential magnetic property, a magnetic particle (e.g., a bead) or compound (e.g., Fe³⁺) can be coupled to the analyte to give it a magnetic property. In some embodiments, a bead coupled to an antibody that selectively binds to an analyte of interest can be decorated with an antibody elected from the group of anti CD71 or CD75. In some embodiments a magnetic compound, such as Fe³⁺, can be couple to an antibody such as those described above. The magnetic particles or magnetic antibodies herein may be coupled to any one or more of the devices herein prior to contact with a sample or may be mixed with the sample prior to delivery of the sample to the device(s). Magnetic particles can also be used to decorate one or more analytes (cells of interest or not of interest) to increase the size prior to performing size-based separation.

A magnetic field used to separate analytes/cells in any of the embodiments herein can be uniform or non-uniform as well as external or internal to the device(s) herein. An external magnetic field is one whose source is outside a device herein (e.g., container, channel, obstacles). An internal magnetic field is one whose source is within a device contemplated herein. An example of an internal magnetic field is one where magnetic particles may be attached to obstacles present in the device (or manipulated to create obstacles) to increase surface area for analytes to interact with to increase the likelihood of binding. Analytes captured by a magnetic field can be released by demagnetizing the magnetic regions retaining the magnetic particles. For selective release of analytes from regions, the demagnetization can be limited to selected obstacles or regions. For example, the magnetic field can be designed to be electromagnetic, enabling turn-on and turn-off off the magnetic fields for each individual region or obstacle at will.

In one embodiment, a method for enriching a fetal nucleated red blood cell is provided comprising contacting said fetal cell with a stabilization composition of the provided invention and enriching said fetal nucleated red blood cell using magnetic-based enrichment.

J. Multiple Modules

Multiple enrichment steps can be used to separate the rare cells (e.g., fnRBC's or placental cells) from non-rare cells, e.g., maternal nucleated red blood cells. In one embodiment, a sample is contacted by a stabilization composition of the provided invention, and the sample is enriched by size-based separation followed by affinity/magnetic separation, and is further enriched for rare cells using fluorescence activated cell sorting (FACS) or selective lysis of a subset of the cells.

In one embodiment, a fluid sample such as a blood sample is contacted by a stabilization composition and is first flowed through one or more size-base separation module. Such modules can be fluidly connected in series and/or in parallel. In one example, the waste (e.g., cells having hydrodynamic size less than 4 microns) are directed into a first outlet and the product (e.g., cells having hydrodynamic size greater than 4 microns) are directed to a second outlet. The product is subsequently enriched using the inherent magnetic property of hemoglobin. The product is modified (e.g., by addition of one or more reagents) such that the hemoglobin in the red blood cells becomes paramagnetic. Subsequently, the product is flowed through one or more magnetic fields. The cells that are trapped by the magnetic field are subsequently analyzed using the one or more methods herein.

One or more of the enrichment modules herein (e.g., size-based separation module(s) and capture module(s)) can be fluidly coupled in series or in parallel with one another. For example a first outlet from a separation module can be fluidly coupled to a capture module. In one embodiment, the separation module and capture module are integrated such that a plurality of obstacles acts both to deflect certain analytes according to size and direct them in a path different than the direction of analyte(s) of interest, and also as a capture module to capture, retain, or bind certain analytes based on size, affinity, magnetism or other physical property.

K. Efficiency of Enrichment

In any of the embodiments herein, the enrichment steps performed can have a specificity and/or sensitivity greater than 50, 60, 70, 80, 90, 95, 96, 97, 98, 99, 99.1, 99.2, 99.3, 99.4, 99.5, 99.6, 99.7, 99.8, 99.9 or 99.95%. The retention rate of the enrichment module(s) herein is such that ≧50, 60, 70, 80, 90, 91, 92, 93, 94, 95, 96, 97, 98, 99, or 99.9% of the analytes or cells of interest (e.g., nucleated cells or nucleated red blood cells or nucleated from red blood cells) are retained. Simultaneously, the enrichment modules are configured to remove ≧50, 60, 70, 80, 85, 90, 91, 92, 93, 94, 95, 96, 97, 98, 99, or 99.9% of all unwanted analytes (e.g., red blood-platelet enriched cells) from a sample.

For example, in one embodiment the analytes of interest are retained in an enriched solution that is less than 50, 40, 30, 20, 10, 9.0, 8.0, 7.0, 6.0, 5.0, 4.5, 4.0, 3.5, 3.0, 2.5, 2.0, 1.5, 1.0, or 0.5 fold diluted from the original sample. In one embodiment, any or all of the enrichment steps increase the concentration of the analyte of interest (e.g., fetal cell), for example, by transferring them from the fluid sample to an enriched fluid sample (sometimes in a new fluid medium, such as a buffer).

### VI. Cell or Cell Component or Cell-Free Nucleic Acid Analysis

A. Conditions

1. Fetal Conditions

Fetal conditions that can be determined based on the compositions, methods, and kits herein include the presence of a fetus and/or a condition of the fetus such as fetal aneuploidy e.g., trisomy 13, trisomy 18, trisomy 21 (Down Syndrome), Klinefelter Syndrome (XXY) and other irregular number of sex or autosomal chromosomes, including monosomy of one or more chromosomes (X chromosome monosomy, also known as Turner's syndrome), trisomy of one or more chromosomes (13, 18, 21, and X), tetrasomy and pentasomy of one or more chromosomes (which in humans is most commonly observed in the sex chromosomes, e.g., XXXX, XXYY, XXXY, XYYY, XXXXX, XXXXY, XXXYY, XYYYY and XXYYY), monoploidy, triploidy (three of every chromosome, e.g., 69 chromosomes in humans), tetraploidy (four of every chromosome, e.g., 92 chromosomes in humans), pentaploidy and multiploidy. Other fetal conditions that can be detected using the methods herein include segmental aneuploidy, such as 1p36 duplication, dup(17)(p11.2p11.2) syndrome, Down syndrome, Pre-eclampsia, Pre-term labor, Edometriosis, Pelizaeus-Merzbacher disease, dup(22)(q11.2q11.2) syndrome, Cat eye syndrome. In one embodiment, the fetal abnormality to be detected is due to one or more deletions in sex or autosomal chromosomes, including Cridu-chat syndrome, Wolf-Hirschhorn syndrome, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, steroid sulfatase deficiency, Kallmann syndrome, Microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c) and 1p36 deletion. In one embodiment, the fetal abnormality is an abnormal decrease in chromosomal number, such as XO syndrome. Conditions associated with chromosome 1, 2, 3, 4, 5, 6, 7, 8, 9, 10, 11, 12, 13, 14, 15, 16, 17, 18, 19, 20, 21, 22, X, or Y can be determined.

2. Cancers

Conditions in a patient that can be detected using the compositions, methods, and kits herein include, for example, infection (e.g., bacterial, viral, or fungal infection), neoplastic or cancer conditions (e.g., acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, breast cancer, bronchi cancer, cervical dysplasia, chronic myelogenous leukemia, colon cancer, epidermoid carcinoma, Ewing's sarcoma, gallbladder cancer, gallstone tumor, giant cell tumor, glioblastoma multiforma, hairy-cell tumor, head cancer, hyperplasia, hyperplastic comeal nerve tumor, in situ carcinoma, intestinal ganglioneuroma, islet cell tumor, Kaposi's sarcoma, kidney cancer, larynx cancer, leiomyomater tumor, liver cancer, lung cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neck cancer, neural tissue cancer, neuroblastoma, osteogenic sarcoma, osteosarcoma, ovarian tumor, pancreas cancer, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, or Wilm's tumor), inflammation, etc.

3. White Blood Cell and Immune Disorders

White blood cell disorders in a patient that can be detected using the compositions, methods, and kits herein include, for example, leukemia, acute lymphoblastic leukemia (ALL), acute myelogenous leukemia (AML), chronic myelogenous leukemia (CML), myelofibrosis, chronic lymphocytic leukemia (CLL), multiple myeloma, infectious mononucleosis, lymphoma, Hodgkin's disease, Non-Hogkin's lymphoma (NHL), low grade NHL, high grade NHL, small lymphocytic lymphoma, follicular lymphoma, large cell lymphoma, Burkitt's lymphoma, lymphoblastic lymphoma, extranodal marginal zone B-cell lymphoma of mucosa-associated lymphoid tissue (MALT), agranulocytosis, and leukopenia.

Other white blood cell disorders that can be detected using the compositions, methods, and kits herein include, for example, basophilic disorders (e.g., basopenia and basophilia); eosinophilic disorders (e.g., eosinopenia. eosinophilia, and idiopathic hypereosinophilic syndrome); lymphocytic leukocytosis (an abnormally high number of lymphocytes in the blood); lymphoctopenia (an abnormally low number of lymphocytes in the blood); monocyte disorders (e.g. monocytosis, monocytopenia, Gaucher's disease, and Niemann-Pick disease); neutropenia (abnormally low number of neutrophils in the blood); neutrophilic leukocytosis (abnormally high number of neutrophils in the blood), leukostasis, leukemoid reactions, leukoerythroblastic reactions

Other immune disorders that can be detected using the compositions, methods, and kits herein include, for example, AIDS, SCID, Chediak-Higashi Syndrome, common variable immunodeficiency, drug allergies, food allergies, insect sting allergies, peanut allergies, penicillin allergy, latex allergies, skin allergies, hives, HTLV, HTLV-1, Hyper-IgE Syndrome, Hyper-IgM Syndrome, leukocyte adhesion defect, primary immune deficiency, selective IgA deficiency, X-Linked agammaglobulinemia, allergic rhinitis, Hay fever, DiGeorge's syndrome, autoimmune lymphoproliferative syndrome, autoimmune neuropathies, lymphadenitis, lymphatic filariasis, POEMS, and thymus cancer.

Other immune disorders that can be detected using the compositions, methods, and kits herein include, for example, autoimmune disease, e.g., acute disseminated encephalomyelitis (ADEM), Addison's disease, alopecia areata, antiphospholipid antibody syndrome (APS), autoimmune hemolytic anemia, autoimmune hepatitis, Coeliac disease, Bullous pemphigoid, Crohns Disease, dermatomyositis, diabetes mellitus type 1, Goodpasture's syndrome, Guillain-Barré syndrome (GBS), Hashimoto's disease, idiopathic thrombocytopenic purpura, Mixed Connective Tissue Disease, myasthenia gravis, narcolepsy, pemphigus vulgaris, pernicious anaemia, polymyositis, primary biliary cirrhosis, Sjögren's syndrome, systemic lupus erythematosus (SLE), multiple sclerosis (MS), Churg-Strauss syndrome, temporal arteritis, ulcerative colitis, vasculitis, Wegener's granulomatosis, Hashimoto's thyroiditis, Graves' disease, and rheumatoid arthritis (RA).

B. Nucleic Acid Analysis

Stabilization compositions of the provided invention can be used in methods for analyzing nucleic acids. In some cases, sample analyses involves performing one or more genetic analyses or detection steps on nucleic acids from the enriched product (e.g., enriched cells or nuclei). Nucleic acids from enriched cells or enriched nuclei that can be analyzed by the methods herein include: double-stranded DNA, single-stranded DNA, single-stranded DNA hairpins, DNA/RNA hybrids, RNA (e.g. mRNA) and RNA hairpins. Examples of genetic analyses that can be performed on enriched cells or nucleic acids include, e.g., SNP detection, STR detection, and RNA expression analysis.

In other cases, genetic analyses or detection steps can be performed on cell-free nucleic acids present in blood samples. In one embodiment, cell-free DNA can be obtained from human blood samples where the cell stabilization compositions of the invention have been added to a blood sample to prevent additional lysis of cells present in the blood. When cell-free DNA is obtained from blood samples from pregnant females, the cell-free nucleic acids are a mixture of maternal and fetal nucleic acids, and the cell-free nucleic acids can be analyzed for fetal genetic conditions (see, e.g., U.S. Pat. No. 7,332,277 and US Patent Application Nos. 20090029377, 20090053719, and 20090087847). In particular, fetal aneuploidy can be determined by analysis of cell-free DNA obtained from maternal serum as described in PCT Publication WO2007092473A2. In particular, cell-free DNA from maternal serum can be analyzed by techniques such as digital PCR and massively parallel DNA sequencing to determine the presence of fetal aneuploidy, as described in U.S. Patent Application No. 20070202525 and Fan H C et al. (2008) PNAS 105:16266-71.

In some embodiments, less than 1 μg, 500 ng, 200 ng, 100 ng, 50 ng, 40 ng, 30 ng, 20 ng, 10 ng, 5 ng, 1 ng, 500 pg, 200 pg, 100 pg, 50 pg, 40 pg, 30 pg, 20 pg, 10 pg, 5 pg, or 1 pg of nucleic acids are obtained from the enriched sample for further genetic analysis. In some cases, about 1-5 μg, 5-10 μg, or 10-100 μg of nucleic acids are obtained from the enriched sample for further genetic analysis.

When analyzing, for example, a sample such as a blood sample from a patient to diagnose a condition such as cancer, the genetic analyses can be performed on one or more genes encoding or regulating a polypeptide, including but not limited to 2AR, Disintegrin, Activator of Thyroid and Retinoic acid receptor (ACTR), ADAM 11, Adipogenesis Inhibitory Factor (ADIF), alpha 6 Integrin subunit, Alpha V integrin subunit, Alpha-Catenin, Amplified In Breast Cancer 1 (AIB1), Amplified In Breast Cancer 3 (AIB3), Amplified In Breast Cancer 4 (AIB4), Amyloid Precursor Protein Secretase (APPS), AP-2 GAMMA, APPS, ATP-Binding Cassette Transporter (ABCT), Placenta-Specific (ABCP), ATP-Binding Cassette Subfamily C member 1 (ABCC1), BAG-1, Basigin (BSG), BCEI, B-Cell Differentiation Factor (BCDF), B-Cell Leukemia 2 (BCL-2), B-Cell Stimulatory Factor-2 (BSF-2), BCL-1, BCL-2-Associated X Protein (BAX), BCRP, Beta 1 Integrin Subunit, Beta 3 Integrin Subunit, Beta 5 Integrin Subunit, Beta-2 Interferon, Beta-Catenin, Bone SIaloprotein (BSP), Breast Cancer Estrogen-Inducible Sequence (BCEI), Breast Cancer REsistance Protein (BCRP), Breast Cancer Type 1 (BRCA1), Breast Cancer Type 2 (BRCA2), BReast CArcinoma Amplified Sequence 2 (BCAS2), Cadherin, Epithelial Cadherin-11, Cadherin-Associated Protein, Calcitonin receptor (CTR), Calcium Placental Protein (CAPL), Calcyclin, CALLA, CAMS, CAPL, Carcinoembryonic Antigen (CEA), CAtenin Alpha 1, Cathepsin B, Cathepsin D, Cathepsin K, Cathepsin L2, Cathepsin O, Cathepsin O1, Cathepsin V, CD10, CD146, CD147, CD24, CD29, CD44, CD51, CD54, CD61, CD66e, CD82, CD87, CD9, CEA, Cellular Retinol-Binding Protein 1 (CRBP1), c-ERBB-2, CK7, CK8, CK18, CK19, CK20, CLAUDIN-7, c-MET, Collagenase-Fibroblast, Collagenase-Interstitial, Collagenase-3, Common Acute Lymphocytic Leukemia Antigen (CALLA), Connexin 26 (Cx26), Connexin 43 (Cx43), Cortactin, COX-2, CTLA-8, CTR, CTSD, Cyclin D1, Cyclooxygenase-2, Cytokeratin 18, Cytokeratin 19, Cytokeratin 8, Cytotoxic T-Lymphocyte-Associated Serine Esterase 8 (CTLA-8), Differentiation-Inhibiting Activity (DIA), DNA Amplified In Mammary Carcinoma 1 (DAM1), DNA Topoisomerase II Alpha, DR-NM23, E-Cadherin, Emmprin, EMS1, Endothelial Cell Growth Factor (ECGR), Platelet-Derived (PD-ECGF), Enkephalinase, Epidermal Growth Factor Receptor (EGFR), Episialin, Epithelial Membrane Antigen (EMA), ER-ALPHA, ERBB2, ERBB4, ER-BETA, ERF-1, Erythroid-Potentiating Activity (EPA), ESR1, Estrogen Receptor-Alpha, Estrogen Receptor-Beta, ETS-1, Extracellular Matrix Metalloproteinase Inducer (EMMPRIN), Fibronectin Receptor, Beta Polypeptide (FNRB), Fibronectin Receptor Beta Subunit (FNRB), FLK-1, GA15.3, GA733.2, Galectin-3, Gamma-catenin, GAP Junction protein (26 kDa), GAP Junction Protein (43 kDa), GAP Junction Protein Alpha-1 (GJA1), GAP Junction Protein Beta-2 (GJB2), GCP1, Gelatinase A, Gelatinase B, Gelatinase (72 kDa), Gelatinase (92 kDa), Gliostatin, Glucocorticoid Receptor Interacting Protein 1 (GRIP1), Glutathione S-Transferase p, GM-CSF, Granulocyte Chemotactic Protein 1 (GCP1), Granulocyte-Macrophage-Colony Stimulating Factor, Growth Factor Receptor Bound-7 (GRB-7), GSTp, HAP, Heat-Shock Cognate Protein 70 (HSC70), Heat-Stable Antigen, Hepatocyte Growth Factor (HGF), Hepatocyte Growth Factor Receptor (HGFR), Hepatocyte-Stimulating Factor III (HSF III), HER-2, HER2/NEU, Hermes Antigen, HET, HHM, Humoral Hypercalcemia Of Malignancy (HHM), ICERE-1, INT-1, Intercellular Adhesion Molecule-1 (ICAM-1), Interferon-Gamma-Inducing Factor (IGIF), Interleukin-1 Alpha (IL-1A), Interleukin-1 Beta (IL-1B), Interleukin-11 (IL-11), Interleukin-17 (IL-17), Interleukin-18 (IL-18), Interleukin-6 (IL-6), interleukin-8 (IL-8), Inversely Correlated With Estrogen Receptor Expression-1 (ICERE-1), KAI1, KDR, Keratin 8, Keratin 18, Keratin 19, KISS-1, Leukemia Inhibitory Factor (LIF), LIF, Lost In Inflammatory Breast Cancer (LIBC), LOT (“Lost On Transformation”), Lymphocyte Homing Receptor, Macrophage-Colony Stimulating Factor, Mage-3, Mammaglobin, Maspin, MC56, M-CSF, MDC, MDNCF, MDR, Melanoma Cell Adhesion Molecule (MCAM), Membrane Metalloendopeptidase (MME), Membrane-Associated Neutral Endopeptidase (NEP), Cysteine-Rich Protein (MDC), Metastasin (MTS-1), MLN64, MMP1, MMP2, MMP3, MMP7, MMP9, MMP11, MMP13, MMP14, MMP15, MMP16, MMP17, Moesin, Monocyte Arginine-Serpin, Monocyte-Derived Neutrophil Chemotactic Factor, Monocyte-Derived Plasminogen Activator InhibitoR, MTS-1, MUC-1, MUC18, Mucin Like Cancer Associated Antigen (MCA), Mucin, MUC-1, Multidrug Resistance Protein 1 (MDR, MDR1), Multidrug Resistance Related Protein-1 (MRP, MRP-1), N-Cadherin, NEP, NEU, Neutral Endopeptidase, NeutrophiL-Activating Peptide 1 (NAP1), NM23-H1, NM23-H2, NME1, NME2, Nuclear Receptor Coactivator-1 (NCoA-1), Nuclear Receptor Coactivator-2 (NCoA-2), Nuclear Receptor Coactivator-3 (NCoA-3), Nucleoside Diphosphate Kinase A (NDPKA), Nucleoside Diphosphate Kinase B (NDPKB), Oncostatin M (OSM), Ornithine Decarboxylase (ODC), Osteoclast Differentiation Factor (ODF), Osteoclast Differentiation Factor Receptor (ODFR), Osteonectin (OSN, ON), Osteopontin (OPN), Oxytocin Receptor (OXTR), p27/kip1, p300/CBP Cointegrator Associate Protein (p/CIP), p53, p9Ka, PAI-1, PAI-2, Parathyroid Adenomatosis 1 (PRAD1), Parathyroid hormone-Like Hormone (PTHLH), Parathyroid Hormone-Related Peptide (PTHrP), P-Cadherin, PD-ECGF, PDGF-b, PEANUT-Reactive Urinary Mucin (PUM), P-Glycoprotein (P-GP), PGP-1, PHGS-2, PHS-2, PIP, Plakoglobin, Plasminogen Activator Inhibitor (Type 1), Plasminogen Activator Inhibitor (Type 2), Plasminogen Activator (TIssue-Type), Plasminogen Activator (Urokinase-Type), Platelet Glycoprotein IIIa (GP3A), PLAU, Pleomorphic AdenomA Gene-Like 1 (PLAGL1), Polymorphic EpitheliaL Mucin (PEM), PRAD1, Progesterone Receptor (PgR), Progesterone Resistance, Prostaglandin Endoperoxide Synthase-2, Prostaglandin G/H Synthase-2, Prostaglandin H Synthase-2 pS2, PS6K, Psoriasin, PTHLH, PTHrP, RAD51, RAD52, RAD54, RAP46, Receptor-Associated Coactivator 3 (RAC3), Repressor OF Estrogen Receptor Activity (REA), S100A4, S100A6, S100A7, S6K, SART-1, Scaffold Attachment Factor B (SAF-B), Scatter Factor (SF), Secreted Phosphoprotein-1 (SPP-1), Secreted Protein, Acidic And Rich In Cysteine (SPARC), Stannicalcin, Steroid Receptor Coactivator-1 (SRC-1), Steroid Receptor Coactivator-2 (SRC-2), Steroid Receptor Coactivator-3 (SRC-3), Steroid Receptor RNA Activator (SRA), Stromelysin-1, Stromelysin-3, Tenascin-C (TN-C), Testes-Specific Protease 50, Thrombospondin I, Thrombospondin II, Thymidine Phosphorylase (TP), Thyroid Hormone Receptor Activator Molecule 1 (TRAM-1), Tight Junction Protein 1 (TJP1), TIMP1, TIMP2, TIMP3, TIMP4, Tissue-Type Plasminogen Activator, TN-C, TP53, tPA, Transcriptional Intermediary Factor 2 (TIF2), Trefoil Factor 1 (TFF1), TSG101, TSP-1, TSP1, TSP-2, TSP2, TSP50, Tumor Cell Collagenase Stimulating Factor (TCSF), Tumor-Associated Epithelial Mucin, uPA, uPAR, Urokinase, Urokinase-Type Plasminogen Activator, Urokinase-Type Plasminogen Activator Receptor (uPAR), Uvomorulin, VAscular Endothelial Growth Factor, Vascular Endothelial Growth Factor Receptor-2 (VEGFR2), Vascular Endothelial Growth Factor-A, Vascular Permeability Factor, VEGFR2, Very Late T-Cell Antigen Beta (VLA-Beta), Vimentin, Vitronectin Receptor Alpha Polypeptide (VNRA), Vitronectin Receptor, Von Willebrand Factor, VPF, VWF, WNT-1, ZAC, ZO-1, or Zonula Occludens-1.

In some cases, a diagnosis is made by comparing results from such genetic analyses with results from similar analyses from a reference sample (one without fetal cells or CTC's, as the case may be). For example, a maternal blood sample enriched for fetal cells can be analyzed to determine the presence of fetal cells and/or a condition in such cells by comparing the ratio of maternal to paternal genomic DNA (or alleles) in control and test samples.

In some embodiments, target nucleic acids from a test sample are amplified and optionally results are compared with amplification of similar target nucleic acids from a non-rare cell population (reference sample). Amplification of target nucleic acids can be performed by any means known in the art. In some cases, target nucleic acids are amplified by polymerase chain reaction (PCR). Examples of PCR techniques that can be used include, but are not limited to, digital PCR, quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), PCR-RFLP/RT-PCR-RFLP, hot start PCR, nested PCR, in situ polony PCR, in situ rolling circle amplification (RCA), bridge PCR, picotiter PCR, digital PCR, and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938.

In any of the embodiments, amplification of target nucleic acids may occur on a bead. In any of the embodiments herein, target nucleic acids may be obtained from a single cell.

In any of the embodiments herein, the nucleic acid(s) of interest can be pre-amplified prior to the amplification step (e.g., PCR). In some cases, a nucleic acid sample may be pre-amplified to increase the overall abundance of genetic material to be analyzed (e.g., DNA). Pre-amplification can therefore include whole genome amplification such as multiple displacement amplification (MDA) or amplifications with outer primers in a nested PCR approach.

In some embodiments amplified nucleic acid(s) are quantified. Methods for quantifying nucleic acids are known in the art and include, but are not limited to, gas chromatography, supercritical fluid chromatography, liquid chromatography (including partition chromatography, adsorption chromatography, ion exchange chromatography, size-exclusion chromatography, thin-layer chromatography, and affinity chromatography), electrophoresis (including capillary electrophoresis, capillary zone electrophoresis, capillary isoelectric focusing, capillary electrochromatography, micellar electrokinetic capillary chromatography, isotachophoresis, transient isotachophoresis and capillary gel electrophoresis), comparative genomic hybridization (CGH), microarrays, bead arrays, high-throughput genotyping such as with the use of molecular inversion probe (MIP), and DNA sequencing.

Quantification of amplified target nucleic acid can be used to determine gene/or allele copy number, gene or exon-level expression, methylation-state analysis, or detect a novel transcript in order to diagnose or condition, i.e. fetal abnormality or cancer.

In one embodiment, analysis involves detecting one or more mutations or SNPs in DNA from e.g., enriched rare cells or enriched rare DNA. Such detection can be performed using, for example, DNA microarrays. Examples of DNA microarrays include those commercially available from Affymetrix, Inc. (Santa Clara, Calif.), including the GeneChip™ Mapping Arrays including Mapping 100K Set, Mapping 10K 2.0 Array, Mapping 10K Array, Mapping 500K Array Set, and GeneChip™ Human Mitochondrial Resequencing Array 2.0. The Mapping 10K array, Mapping 100K array set, and Mapping 500K array set analyze more than 10,000, 100,000 and 500,000 different human SNPs, respectively. SNP detection and analysis using GeneChip™ Mapping Arrays is described in part in Kennedy, G. C., et al., Nature Biotechnology 21, 1233-1237, 2003; Liu, W. M., Bioinformatics 19, 2397-2403, 2003; Matsuzaki, H., Genome Research 3, 414-25, 2004; and Matsuzaki, H., Nature Methods, 1, 109-111, 2004 as well as in U.S. Pat. Nos. 5,445,934; 5,744,305; 6,261,776; 6,291,183; 5,799,637; 5,945,334; 6,346,413; 6,399,365; and 6,610,482, and EP 619 321; 373 203. In some embodiments, a microarray is used to detect at least 5, 10, 20, 50, 100, 200, 500, 1,000, 2,000, 5,000 10,000, 20,000, 50,000, 100,000, 200,000, or 500,000 different nucleic acid target(s) (e.g., SNPs, mutations or STRs) in a sample.

Methods for analyzing chromosomal copy number using mapping arrays are disclosed, for example, in Bignell et al., Genome Res. 14:287-95 (2004), Lieberfarb, et al., Cancer Res. 63:4781-4785 (2003), Zhao et al., Cancer Res. 64:3060-71 (2004), Nannya et al., Cancer Res. 65:6071-6079 (2005) and Ishikawa et al., Biochem. and Biophys. Res. Comm., 333:1309-1314 (2005). Computer implemented methods for estimation of copy number based on hybridization intensity are disclosed in U.S. Publication Application Nos. 20040157243; 20050064476; and 20050130217.

In preferred aspects, mapping analysis using fixed content arrays, for example, 10K, 100K or 500K arrays, preferably identify one or a few regions that show linkage or association with the phenotype of interest. Those linked regions may then be more closely analyzed to identify and genotype polymorphisms within the identified region or regions, for example, by designing a panel of MIPs targeting polymorphisms or mutations in the identified region. The targeted regions may be amplified by hybridization of a target specific primer and extension of the primer by a highly processive strand displacing polymerase, such as phi29 and then analyzed, for example, by genotyping.

Analytical techniques that can be used with the compositions, methods, and kits of the provided invention include, for example, Western blotting, Southern blotting, SDS-PAGE, gel electrophoresis, Northern blotting, comparative genomic hybridization (CGH), chromosomal microarray analysis (CMA), expression profiling, DNA microarray, high-density oligonucleotide microarray, whole-genome RNA expression array, peptide microarray, polymerase chain reaction (PCR), digital PCR (dPCR), reverse transcription PCR, quantitative PCR (Q-PCR), single marker qPCR, real-time PCR, nCounter Analysis (Nanostring technology), enzyme-linked immunosorbent assay (ELISA), genome sequencing, de novo sequencing, pyrosequencing, polony sequencing, copy number variation (CNV) analysis sequencing, small nucleotide polymorphism (SNP) analysis, immunohistochemistry (IHC), immunoctyochemistry (ICC), mass spectrometry, tandem mass spectrometry, in-situ hybridization, either DNA or RNA fluorescent in-situ hybridization (FISH), and chromogenic in-situ hybridization (CISH).

In one embodiment, a method for diagnosing a fetal condition is provided comprising contacting a fetal cell from a maternal blood sample with a stabilization composition of the provided invention, enriching said fetal cell by size-based separation, density gradient centrifugation, or red blood cell lysis, performing FISH, and determining said fetal condition based on said FISH. In one embodiment the fetal condition is aneuploidy.

DNA Sequencing

In one embodiment, a sample (e.g., a maternal blood sample) comprising cell-free fetal DNA is contacted by a stabilization composition of the provided invention, and DNA sequencing is used to determine the presence or absence of a fetal condition using the DNA. DNA sequencing techniques that can be used in the methods of the provided invention include, for example, Helicos True Single Molecule Sequencing (tSMS) (Harris T. D. et al. (2008) Science 320:106-109); 454 sequencing (Roche) (Margulies M. et al. (2005) Nature 437:376-380); SOLiD sequencing (Applied Biosystems); SOLEXA sequencing (Illumina); single molecule, real-time (SMRT™) technology of Pacific Biosciences; nanopore sequencing (Soni G V and Meller A. (2007) Clin Chem 53:1996-2001); and sequencing using a chemical-sensitive field effect transistor (chemFET) array (as described in US Patent Application Publication No. 20090026082). The tSMS, SOLiD sequencing, SOLEXA sequencing, and SMRT sequencing are sequencing by synthesis methods. Nanopore sequencing and sequencing using chemical-sensitive field effect transistor (chemFET) arrays does not involve synthesis.

In one embodiment, high-throughput sequencing involves the use of technology available by Helicos BioSciences Corporation (Cambridge, Mass.) such as the True Single Molecule Sequencing (tSMS) method. tSMS can allow for sequencing the entire human genome in up to 24 hours. This fast sequencing method also allows for detection of a SNP/nucleotide in a sequence in substantially real time or real time. tSMS does not require a preamplification step prior to hybridization. tSMS is described in part in US Publication Application Nos. 20060024711, 20060024678, 20060012793, 20060012784, and 20050100932.

In one embodiment, high-throughput sequencing involves the use of technology available by 454 Lifesciences, Inc. (Branford, Conn.) such as the PicoTiterPlate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours. In 454 sequencing, adapters are ligated to the ends of sheared DNA fragments. The fragments are attached to individual capture beads, the fragments are PCR amplified within droplets of an oil-water emulsion. Beads with clonally amplified DNA are individually captured in pico-liter sized wells. Pyrosequencing is performed on each DNA fragment in parallel. Methods for using bead amplification followed by fiber optics detection are described in (Margulies M. et al. (2005) Nature 437:376-380) and in US Publication Application Nos. 20020012930, 20030068629, 20030100102, 20030148344, 20040248161, 20050079510, 20050124022, and 20060078909.

In some embodiments, PCR-amplified single-strand nucleic acid is hybridized to a primer and incubated with a polymerase, ATP sulfurylase, luciferase, apyrase, and the substrates luciferin and adenosine 5′ phosphosulfate (e.g., pyrosequencing). Next, deoxynucleotide triphosphates corresponding to the bases A, C, G, and T (U) are added sequentially. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process repeats until the entire sequence is determined. In one embodiment, pyrosequencing analyzes DNA methylations, mutation and SNPs. In another embodiment, pyrosequencing also maps surrounding sequences as an internal quality control. Pyrosequencing analysis methods are known in the art.

In one embodiment, high-throughput sequencing is performed using Clonal Single Molecule Array (SOLEXA, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication Application Nos. 20040106110; 20030064398; 20030022207; and Constans, A., The Scientist 2003, 17(13):36. Genetic material e.g., gDNA is obtained using methods known in the art or disclosed herein. The genetic material e.g., gDNA is randomly fragmented. The randomly fragmented gDNA is ligated with adapters on both ends. The genetic material, e.g., ssDNA is bound randomly to inside surface of a flow cell channels. Unlabeled nucleotides and enzymes are added to initiate solid phase bridge amplification. The above step results in genetic material fragments becoming double stranded and bound at either end to the substrate. The double stranded bridge is denatured to create to immobilized single stranded genomic DNA (e.g., ssDNA) sequencing complementary to one another. The above bridge amplification and denaturation steps are repeated multiple times (e.g., at least 10, 50, 100, 500, 1,000, 5,000, 10,000, 50,000, 100,000, 500,000, 1,000,000, 5,000,000 times) such that several million dense clusters of dsDNA (or immobilized ssDNA pairs complementary to one another) are generated in each channel of the flow cell. The first sequencing cycle is initiated by adding all four labeled reversible terminators, primers, and DNA polymerase enzyme to the flow cell. This sequencing-by-synthesis (SBS) method utilizes four fluorescently labeled modified nucleotides that are especially created to posses a reversible termination property, which allow each cycle of the sequencing reaction to occur simultaneously in the presence of all four nucleotides (A, C, T, G). In the presence of all four nucleotides, the polymerase is able to select the correct base to incorporate, with the natural competition between all four alternatives leading to higher accuracy than methods where only one nucleotide is present in the reaction mix at a time which require the enzyme to reject an incorrect nucleotide. All unincorporated labeled terminators are then washed off. A laser is applied to the flow cell. Laser excitation captures an image of emitted fluorescence from each cluster on the flow cell. A computer program product comprising a computer executable logic records the identity of the first base for each cluster. Before initiated the next sequencing step, the 3′ terminus and the fluorescence from each incorporated base are removed.

Subsequently, a second sequencing cycle is initiated, just as the first was by adding all four labeled reversible terminators, primers, and DNA polymerase enzyme to the flow cell. A second sequencing read occurs by applying a laser to the flow cell to capture emitted fluorescence from each cluster on the flow cell which is read and analyzed by a computer program product that comprises a computer executable logic to identify the first base for each cluster. The above sequencing steps are repeated as necessary to sequence the entire gDNA fragment. In some cases, the above steps are repeated at least 5, 10, 50, 100, 500, 1,000, 5,000, to 10,000 times.

In one embodiment, sequence analysis of the rare cell's genetic material may include a four-color sequencing by ligation scheme (degenerate ligation) (e.g., SOLiD sequencing), which involves hybridizing an anchor primer to one of four positions. Then an enzymatic ligation reaction of the anchor primer to a population of degenerate nonamers that are labeled with fluorescent dyes is performed. At any given cycle, the population of nonamers that is used is structure such that the identity of one of its positions is correlated with the identity of the fluorophore attached to that nonamer. To the extent that the ligase discriminates for complementarity at that queried position, the fluorescent signal allows the inference of the identity of the base. After performing the ligation and four-color imaging, the anchor primer:nonamer complexes are stripped and a new cycle begins. Methods to image sequence information after performing ligation are known in the art.

C. Quantitative Evaluation

In one embodiment, the provided invention involves the analysis of maternal blood for a genetic condition, wherein the mixed fetal and maternal nucleic acids in a sample, e.g., a maternal blood, are analyzed to distinguish a fetal mutation or genetic abnormality from the background of the maternal nucleic acids. A nucleic acid sample containing nucleic acid from both the mother and the fetus can be analyzed to distinguish a genetic condition present in a minor fraction of the nucleic acids, which represents the fetal nucleic acids. The method employs “digital analysis,” in which target nucleic acid in the sample is enumerated, that is, 0, 1, 2, 3, etc. A control sequence is used to distinguish an abnormal increase in the target sequence, e.g., a trisonomy. Thus there is a differential detection of target sequences, one of which is chosen to represent a normal genotype present in both mother and offspring, and one of which is chosen for detection of an abnormal genotype in the offspring, where the target sequence in the offspring will be different from that of the mother, e.g. in trisomy.

Techniques for using digital analysis for diagnosing fetal conditions using PCR amplification are described, for example, in US Patent Application Publication No. 20070202525 and PCT Publication Nos. WO2009013492A1 and WO2009019455A2, which are herein incorporated by reference in their entireties. Techniques for digital analysis for diagnosing fetal conditions using massively parallel sequencing techniques that use nucleic acid amplification or DNA synthesis are described, for example, in US Patent Application Nos. 20050221341, 20060046258, and 20090029377, which are herein incorporated by reference in their entireties.

Digital PCR (dPCR) can be used to detect aneuploidy in a fetus using a maternal sample. In order to determine fetal aneuploidy by digital PCR, a maternal blood sample is obtained. The maternal blood sample can be collected into a container containing an anticoagulant, e.g., heparin. A composition of the provided invention, for example, a concentrated form of Composition A, Composition B, Composition C, or Composition D, can be mixed with the maternal blood sample to stabilize cells, e.g., maternal blood cells. Cell-free DNA is isolated from the sample and is diluted (e.g., into wells of a multiwell plate) such that only 0 or 1 DNA molecule is in a well. Primers for the chromosome of interest (e.g., chromosome 21) and a control chromosome are used to amplify DNA, and the number of wells with PCR product is enumerated. The presence or absence of aneuploidy (e.g., Down syndrome) can be determined by statistical analysis (see, e.g., US Patent Publication 20070202525).

In one embodiment, a method for diagnosing a fetal condition is provided comprising obtaining a maternal blood sample comprising cell-free DNA, stabilizing a maternal blood cell in said maternal blood sample by contacting said maternal blood cell with a stabilization composition of the provided invention, isolating DNA comprising cell-free fetal DNA from said sample, sequencing said cell-free DNA, and determining the presence of absence of a fetal condition based on said sequencing. The DNA sequencing techniques described above can be used in the sequencing.

In another embodiment, a method for diagnosing aneuploidy is provided comprising obtaining a maternal blood sample comprising cell-free DNA, stabilizing a maternal blood cell is said maternal blood by contacting said maternal blood cell with a stabilization composition of the provided invention, isolating DNA comprising cell-free DNA from said sample, sequencing said cell-free DNA, enumerating sequences from a chromosome suspected of being aneuploid in the fetus and euploid in the mother, enumerating sequences from a chromosome suspected of being euploid in the fetus and the mother, and determining the presence or absence of said aneuploidy based on said enumeration of sequences.

D. Nanostring nCounter System

Nucleic acids in a sample can be digitally analyzed without amplification or synthesis steps using the target nucleic acids as a template using the Nanostring nCounter system.

The Nanostring nCounter system is technology that can capture and count specific nucleic acids in a complex mixture. In general, use of the nCounter system involves mixing nucleic acids with nanoreporters, which can be pairs of capture probes and coded reporter probes, hybridizing the probe pairs to target sequences, washing away excess probe, binding the hybridized targets to a surface using the capture probe, altering the orientation of the captured molecules to facilitate observation of the coded reporter probes, observing the coded reporter probes by, e.g., single molecular imaging, and enumerating targets based on the coded reporter probes. Enumerating targets in a maternal sample can be used to diagnosis a fetal chromosomal abnormality. Reporter probes, systems and methods for analyzing reporter probes, and methods and computer systems for identifying target specific sequences are described in PCT Publication Nos. WO2007076128, WO2007076129, WO2007076132, WO2007139766, and WO2008124847, and in Geiss G K et al. (2008) Nature Biotechnology 26: 317-325, each of which is herein incorporated by reference in their entireties.

In one embodiment, a method of diagnosing a fetal condition is provided comprising obtaining a maternal blood sample, contacting said maternal blood sample with a stabilization composition of the provided invention, isolating DNA (e.g., cell-free DNA) from said sample, enumerating the DNA using coded reporter probes, and determining the presence or absence of said fetal condition based on said enumerating. Coded reporter probes can be generated that anneal to DNA sequences from a chromosome of interest (e.g., chromosome 21) suspected of being aneuploid in a fetus and euploid in a mother and a control chromosome (e.g., chromosome 1) suspected of being euploid in a fetus and a mother.

E. Fetal Cell Identification

The stabilizing compositions of the provided invention can be used for the purpose of identifying and/or enumerating fetal cells. In one embodiment, a sample (e.g., a maternal blood sample) is contacted by a stabilization composition of the provided invention, and cells (e.g., fetal cells) in the sample are identified and/or enumerated. Identifying and/or enumerating fetal cells can comprise detecting protein or transcript expression from one or more genes in one or more fetal cells, wherein the one or more genes is hPL, CHS2, KISS1, GDF15, CRH, TFP12, CGB, LOC90625, FN1, COL1A2, PSG9, HBE, AFP, APOC3, SERPINC1, AMBP, CPB2, ITIH1, APOH, HPX, beta-hCG, AHSG, APOB, or J42-4d. In another aspect this invention provides a method for identifying an fnRBC comprising detecting transcript or protein expression of a HBE, AFP, AHSG, or J42-4d gene. In one embodiment the detecting comprises using at least two primers and at least on probe that anneal to a cDNA generated from a transcript expressed by said HBE, AFP, AHSG, or J42-4d gene.

In another aspect this invention provides a method for identifying a trophoblast comprising detecting transcript or protein expression of a KISS1, LOC90625, AFP, hPL, beta-hCG, or FN1 gene. In one embodiment the detecting comprises using at least two primers and at least one probe that anneals to a cDNA generated from a transcript expressed by said KISS1, LOC90625, AFP, hPL, beta-hCG, or FN1 gene.

In another aspect this invention provides a method for identifying a fetal cell in a maternal sample comprising detecting transcript or protein expression by a cell of one or more of the KISS1, LOC90625, FN1, or AHSG genes to distinguish said fetal cell from a maternal cell.

In another aspect this invention provides a method for identifying a fetal cell in a sample comprising detecting transcript or protein expression by a cell of three or more of the hPL, KISS1, LOC90625, FN1, PSG9, HBE, AFP, beta-hCG, AHSG or J42-4d genes to distinguish said fetal cell from a maternal cell.

In one embodiment, a method of identifying a fetal cell is provided comprising obtaining a maternal blood sample comprising a fetal cell, contacting said fetal cell with a stabilization composition of the provided invention, and identifying said fetal cell using a probe that detects expression of one or more of the genes hPL, CHS2, KISS1, GDF15, CRH, TFP12, CGB, LOC90625, FN1, COL1A2, PSG9, HBE, AFP, APOC3, SERPINC1, AMBP, CPB2, ITIH1, APOH, HPX, beta-hCG, AHSG, APOB, or J42-4d.

F. Methods

In one embodiment, a method for diagnosing a fetal condition is provided including contacting a maternal blood sample with a stabilization composition of the provided invention and analyzing one or more cells or cellular components (e.g., cell-free DNA) from said sample to diagnosis said fetal condition.

The method for diagnosing a fetal condition can include enriching fetal cells from the sample using size-based separation, selective red blood cell lysis, or density gradient centrifugation. The method can include contacting a sample with a lysis reagent that selectively lysis enucleated red blood cells over nucleated red blood cells. The method can include an antibody-based enrichment step. The analyzing can include performing fluorescent in-situ hybridization (FISH) on DNA or DNA sequencing. The DNA sequencing can be on cell-free DNA.

DNA sequencing can be used to determine fetal aneuploidy using a sample from maternal source. Cell-free DNA from maternal blood can be sequenced using a method described herein (e.g., SOLEXA sequencing). Two or more genomic DNA regions can be sequenced, and the regions can be on the same or different chromosomes. For example, one of the regions can be from a chromosome that is suspected of being aneuploid in a fetus and the other chromosome region can be from a chromosome known to be or suspected to be euploid in a fetus. The number of sequenced fragments from each region can be enumerated by mapping the sequence reads onto human chromosomes and quantitating the number of reads mapping to particular chromosomes using bioinformatic analysis and the sequence information available for the human genome. For example, the ratio of the enumerated fragments from different chromosomes can be used to determine whether the fetus has aneuploidy.

### Examples

### Example 1

### Composition A

Table 1 lists components of Composition A, a composition of the provided invention.

TABLE 1
Composition A

| | Final Concentration |
|---|---|
| Component | (1x) (mM) |
| ddH20 | |
| sodium citrate | 11 |
| adenosine | 0.37 |
| theophylline | 1.5 |
| dipyridamole | 0.02 |
| glycine | 0.50 |
| NAC | 0.50 |
| glutamine | 0.50 |
| D-mannitol | 6.00 |
| PBS | |
| Formaldehyde | 0.04% |
| Potassium dichromate | 0.025% |
| (aged for 3 weeks) | |

### Example 2

### Composition B

Composition B (see Table 2) contains components that can be used to fix white blood cells.

TABLE 2
Composition B

| | Final Concentration |
|---|---|
| Component | (1x) (mM) |
| ddH20 | |
| sodium citrate | 11 |
| adenosine | 0.37 |
| theophylline | 1.5 |
| dipyridamole | 0.02 |
| glycine | 0.50 |
| NAC | 0.50 |
| glutamine | 0.50 |
| D-mannitol | 5.99 |
| PBS | |
| PEG-400 | 1.25 |
| Imidazolidinyl urea | 1.29 |
| (IDU) | |
| Diazolidinyl urea (DU) | 7.19 |

### Example 3

### Anticoagulants Affect Fetal Cell Number

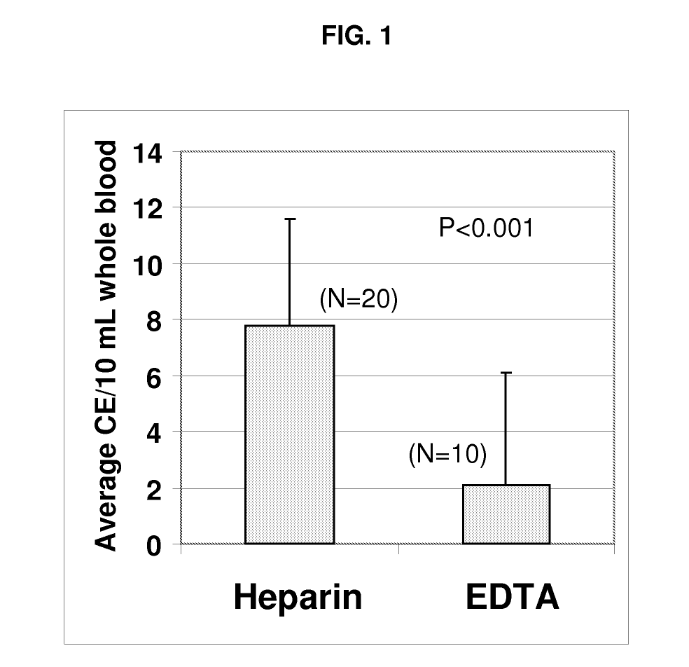

The number of fetal cells in solutions containing EDTA or heparin were compared (FIG. 1). Pre-procedural blood samples (10 ml per condition) were drawn into either sodium heparin tubes or EDTA tubes. Samples were processed within 2 hrs after blood draw. Briefly, whole blood samples were centrifuged to separate blood cells and plasma. Fetal gender was determined by digital PCR using plasma. Blood cell pellets were washed 5 times in PBS to remove cell-free DNA. DNA was then extracted from washed blood cells with a Qiagen column. The fetal cell number in samples with a male fetus was enumerated by digital PCR.

### Example 4

### A Stabilization Composition Enhances Fetal Cell Stability

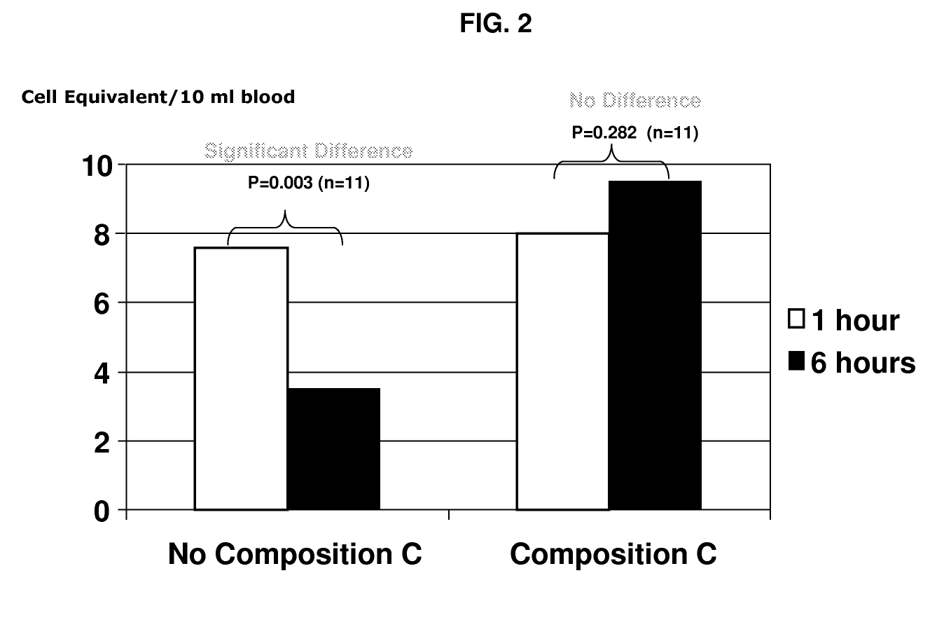

More fetal cells are stabilized over 6 hr in a solution containing Composition C (Table 3; FIG. 2) compared to a solution lacking Composition C. Blood samples were drawn into lithium heparin tubes. Composition C was added to a set of 10 mL blood samples within 30 min, while another set of 10 mL samples from the same patient did not receive Composition C. Sample pairs (with or without Composition C) were processed either at 1 hour or at 6 hours at room temperature. Fetal cell number was enumerated by digital PCR.

TABLE 3
Composition C

| | Final Concentration |
|---|---|
| Component | (1x) (mM) |
| Sodium citrate | 11.0 |
| Theophylline | 1.5 |
| Adenosine | 0.37 |
| Dipyridamole | 0.0198 |
| Glycine | 0.25 |
| NAC | 0.25 |
| Glutamine | 0.25 |
| Dextrose | 12.2 |

### Example 5

### Composition A Keeps Fetal Cells Intact for Up to 96 hr

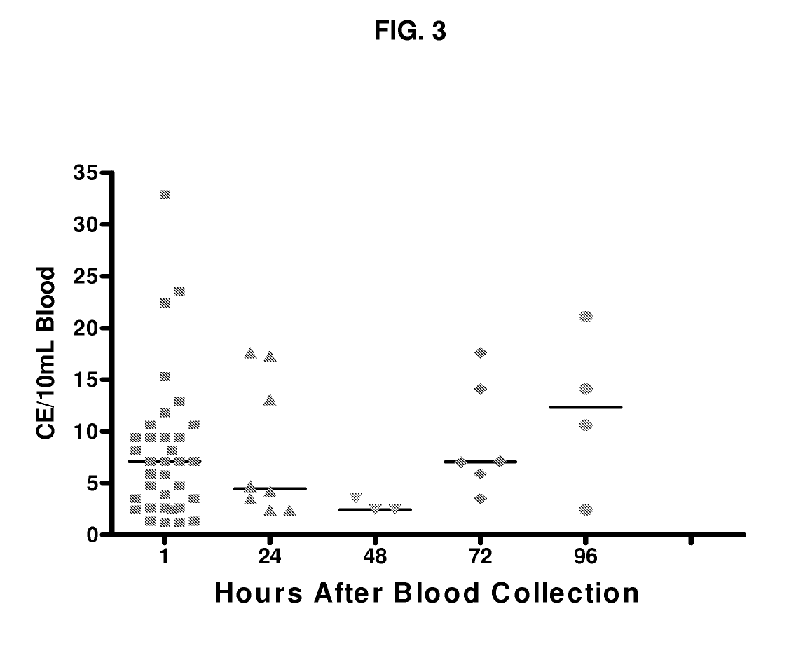

FIG. 3 depicts numbers of cell equivalents in 10 mL blood at 1, 24, 48, 72, and 96 hr after collection and in Composition A. Each dot corresponds to a sample that was checked for fetal cell content at a certain point in time. Each sample was analyzed for fetal cell content at 1 hour. Then, some of these samples were re-analyzed for fetal cell content at 24 hrs, 48 hrs, 72 hrs, or 96 hrs.

### Example 6

### Stabilization of Fetal Cells

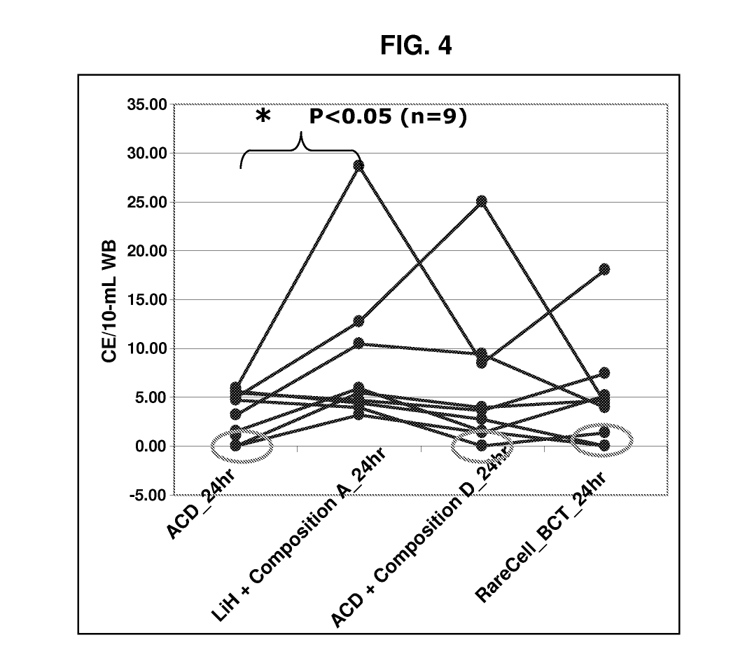

FIG. 4 shows a comparison of number of fetal cells at 24 hr after blood draw in citric acid, sodium citrate, and dextrose (ACD; BD-Biosciences), lithium heparin+Composition A, ACD+Composition D, and RareCell™ BCT (Streck Innovations). The composition of Composition D is provided in Table 4.

TABLE 4
Composition D

| | Final Concentration |
|---|---|
| Component | (1x) (mM) |
| ddH20 | |
| adenosine | 0.37 |
| theophylline | 1.5 |
| dipyridamole | 0.02 |
| glycine | 0.50 |
| NAC | 0.50 |
| glutamine | 0.50 |
| D-mannitol | 6.00 |
| PBS | |
| Formaldehyde | 0.04% |
| Potassium | 0.025% |
| dichromate, aged | |

For the lithium heparin+Composition A sample, maternal blood was drawn into a lithium heparin tube. Composition A was added to a 1× concentration within 1 hr.

For the ACD+Composition D sample, maternal blood was drawn into ACD tubes. Composition D was added within 4-8 hr.

For the Rare-Cell BCT sample, maternal blood was drawn directly into 10 mL Rare-Cell BCT.

FIG. 4 shows the number of cell equivalents from 10 mL whole blood at 24 hr. Nine samples were analyzed. Each sample was split into 4 smaller samples (one 40 mL sample into four 10 mL samples). Each of the smaller samples was mixed with a different cocktail of compounds. Dots that are connected by a line correspond to four “sub-samples” that came from the same original sample.

Statistics were by one-way ANOVA of repeated measurements.

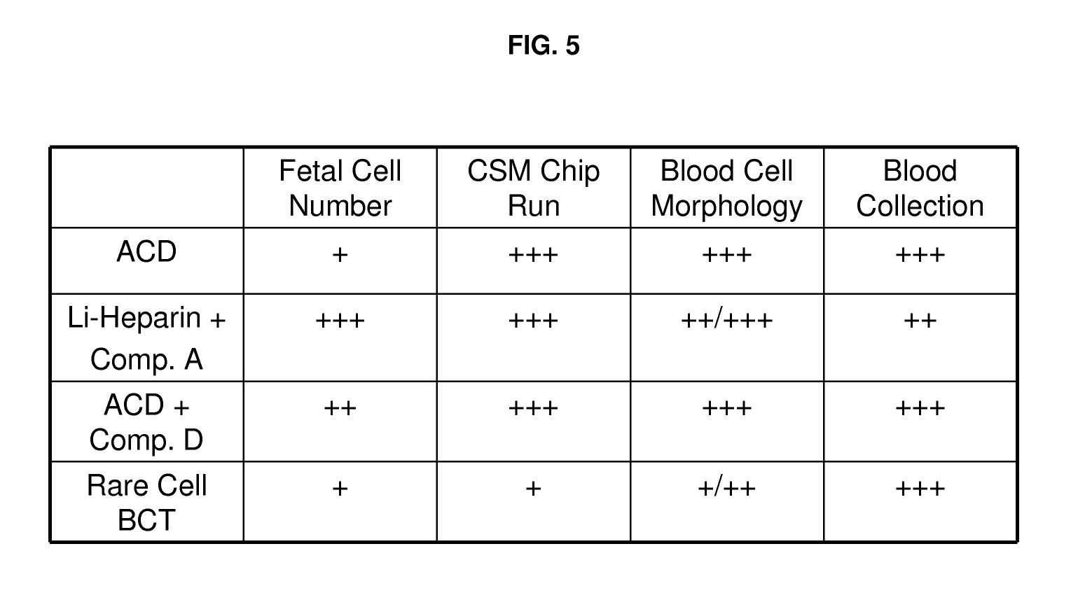

FIG. 5 depicts a rating summary of fetal cell stabilization compositions. “CSM (cell separation module) chip run” refers to the quality of the chip run (with respect to, e.g., clogs, rate, fetal cell stability). More (+) indicates a “better” CSM chip run. “Blood collection” refers the practical convenience of using the composition.

### Example 7

### Cell Stabilization Provides Fetal Cell Preservation

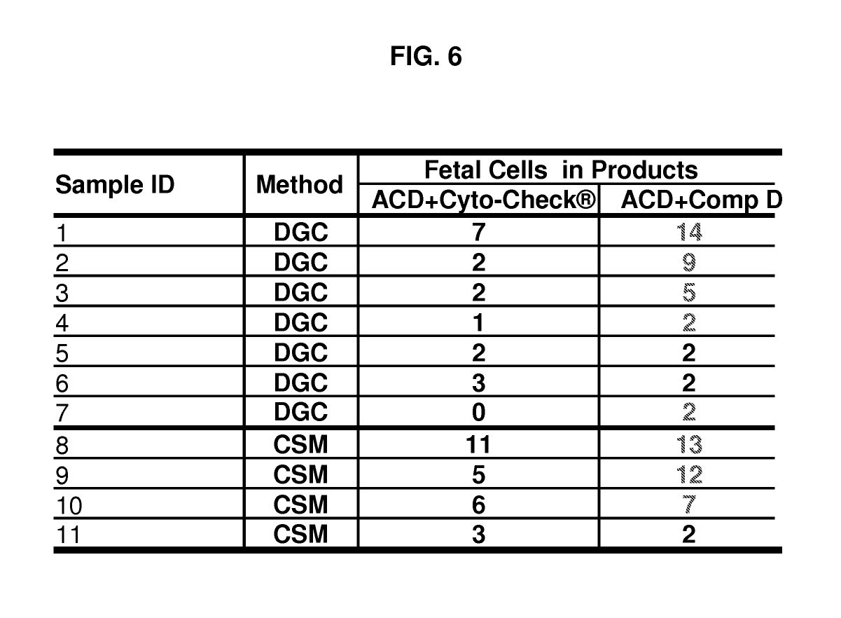

Maternal blood samples were mixed with ACD+CytoCheck® or ACD+Composition D. Fetal cells were enriched using density gradient centrifugation (DGC) (see protocol in Example 14) or size-based separation through a two dimensional array of obstacles (cell separation module; CSM). Fetal cells were counted. In 8 out of 11 samples, more fetal cells were observed in the samples with Composition D (FIG. 6).

### Example 8

### Blood Cell Morphology in ACD+Composition D at 76 hrs

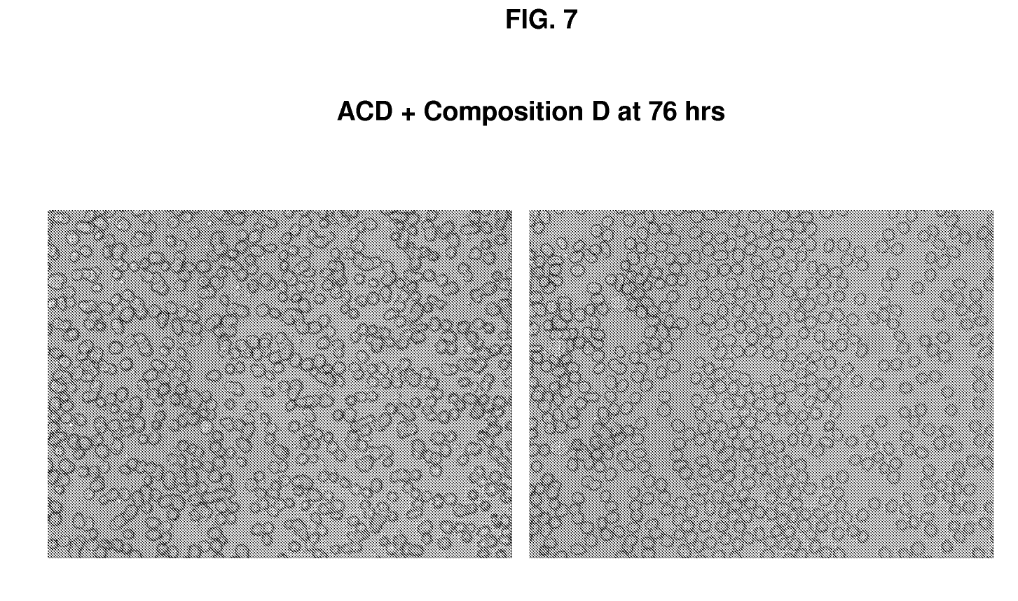

FIG. 7 illustrates blood cell morphology in ACD+Composition D at 76 hrs for two different samples. Overall, blood cells are intact. Some cell membranes show roughness. There is some white blood cell degradation.

### Example 9

### Intact Fetal Cells are Recovered After Size-Based Separation Using Composition C

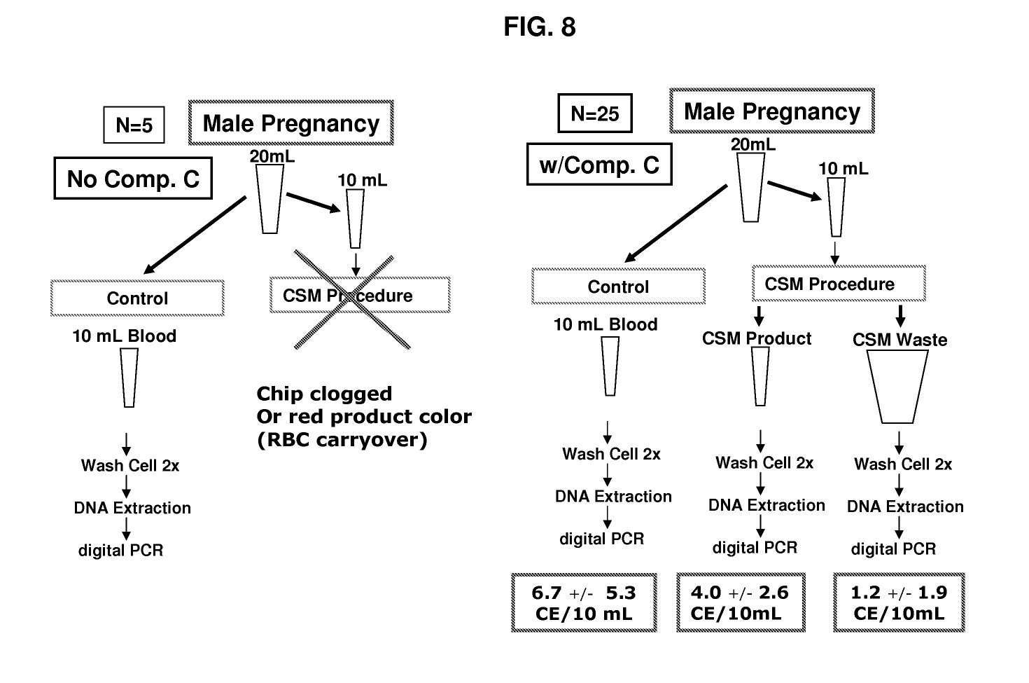

Maternal blood samples from women carrying a male fetus were mixed with or without Composition C. The samples without Composition C clogged the cell separation device or had RBC carryover (FIG. 8).

The number of cells in the control sample mixed with Composition C was determined to be 6.7±5.3 CE/10 mL. Another portion of the sample mixed with Composition C was applied to the size-based separation device. The product and waste were collected. Cells in the control sample, the product, and the waste were washed 2×, DNA was extracted, and digital PCR was performed. Cell recovery was 6.7±5.3 CE/10 mL for the control sample, 4.0±2.6 CE/10 mL for the product, and 1.2±1.9 CE/10 mL for the waste.

### Example 10

### Compositions of the Provided Invention

TABLE 5
Stabilization compositions of the provided invention (1X concentrations provided).

| Composition E | Composition F | Composition G | Composition H | Composition I | Composition J |
|---|---|---|---|---|---|
| EDTA | Glucose | Glucose | Glucose | EDTA | EDTA |
| 0.5 mM | 0.5 mM | 1 mM | 2 mM | 0.5 mM | 0.5 mM |
| Sodium citrate | Glutaraldehyde | Sodium heparin | Sodium heparin | Sorbitol | Ascorbic acid |
| 5 mM | 0.2% | 10 IU/mL | 10 IU/mL | 0.5 mM | 0.5 mM |
| Adenosine | Sodium citrate | Adenonsine | Sodium citrate | NaCl | Adenonsine |
| 1.25 mM | 2.5 mM | 1.0 mM | 15 mM | 10 mM | 1.0 mM |
| Dipyridamole | Glutathione | Glutathione | Adenosine | Formalin | Glutathione |
| 0.1 mM | 0.75 mM | 2.5 mM | 1.0 mM | 0.05% | 2.5 mM |
| Theophylline | Glycine | Glutamine | Dipyridamole | HEPES | Glycine |
| 4 mM | 2 mM | 1.5 mM | 1 mM | 25 mM pH 7.4 | 2 mM |
| Glutathione | HEPES | HEPES | Theophylline | Zinc citrate | sodium citrate |
| 2.5 mM | 10 mM, pH 7.6 | 20 mM, pH 7.4 | 0.25 mM | 10 mM | 15 mM |
| Sorbitol | Oxalate | ZnSO₄ | glutaraldehyde | Imidazole | PBS |
| 0.15 mM | 0.1 mM | 0.5 mM | 0.05% | 1 mM | pH 7.2 |
| PBS | PEG-2001% | glutaraldehyde | EGTA | glutaraldehyde | glutaraldehyde |
| pH 7.2 | | 0.2% | 0.5 mM | 0.02% | 0.1% |
| PEG-600 | | | PEG-600 | PEG-600 | |
| 1% | | | 1% | 1% | |

### Example 11

### Compositions of the Provided Invention

TABLE 6
Additional stabilization compositions of the provided invention (1X concentrations provided).

| Composition K | Composition L | Composition M | Composition N | Composition O | Composition P |
|---|---|---|---|---|---|
| Sodium heparin | Sodium heparin | EDTA | EDTA | EGTA | EGTA |
| 15 IU/mL | 15 IU/mL | 0.25 mM | 0.25 mM | 0.5 mM | 0.5 mM |
| Theophylline | d-mannitol | d-mannitol | Sucrose | d-mannitol | Disodium |
| 1 mM | 10 mM | 10 mM | 1.5 mM | 15 mM | cromoglycate |
| | | | | | 0.5 mM |
| Mannose | Formaldehyde | Formaldehyde | PEG-1000 | PEG-1000 | Sorbitol |
| 1 mM | 0.05% | 0.1% | 1% | 1% | 0.2 mM |
| Imidazole | Imidazole | Imidazole | Tryptophan | Tryptophan | Tryptophan |
| 0.65 mM | 1.3 mM | 1.3 mM | 0.4 mM | 0.4 mM | 0.4 mM |
| thiodipropionic | sodium | thiodipropionic | NAC | NAC | Tris |
| acid | bisulfite | acid | 1 mM | 2.0 mM | 20 mM pH 8.1 |
| 1.5 mM | 1 mM | 1.5 mM | | | |
| Disodium | Disodium | Disodium | Disodium | Disodium | PEG-1000 |
| cromoglycate | cromoglycate | cromoglycate | cromoglycate | cromoglycate | 1% |
| 1 mM | 0.5 mM | 0.5 mM | 1 mM | 1 mM | |
| Sorbitol | Sucrose | Sucrose | Tris | Sorbitol | |
| 0.2 mM | 1 mM | 1.5 mM | 20 mM pH 8.1 | 0.2 mM | |
| PMSF | Sodium fluoride | Sodium fluoride | | Tris | |
| 0.5 mM | 0.5 mM | 0.5 mM | | 20 mM pH 8.1 | |
| Tris | Tris | Tris | | | |
| 25 mM pH 7.9 | 25 mM pH 7.9 | 25 mM pH 7.9 | | | |

### Example 12

### Compositions of the Provided Invention

TABLE 7
Additional stabilization compositions of the provided invention
(1X concentrations provided).

| Composition Q | Composition R | Composition S |
|---|---|---|
| Sodium Citrate | Sodium Citrate | Sodium Citrate |
| 11 mM | 11 mM | 11 mM |
| Adenosine | Adenosine | Adenosine |
| 0.37 mM | 0.37 mM | 0.37 mM |
| Theophylline | Theophylline | Theophylline |
| 1.5 mM | 1.5 mM | 1.5 mM |
| Dipyridamole | Dipyridamole | Dipyridamole |
| 0.02 mM | 0.0198 mM | 0.0198 mM |
| Glycine | Glycine | Glycine |
| 0.25 mM | 0.25 mM | 0.25 mM |
| NAC | NAC | NAC |
| 0.25 mM | 0.25 mM | 0.25 mM |
| Glutamine | Glutamine | Glutamine |
| 0.25 mM | 0.25 mM | 0.25 mM |
| Formaldehyde (36.5% | Formaldehyde (36.5% | Formaldehyde (36.5% |
| stock) | stock) | stock) |
| 0.04% | 0.08% | 0.04% |
| Potassium dichromate, | Potassium dichromate, | Potassium dichromate, |
| aged (5% stock) | aged (5% stock) | aged (5% stock) |
| 0.025% | 0.05% | 0.025% |
| | Beta-cyclodextrin (beta- | Beta-cyclodextrin |
| | CD) | (beta-CD) |
| | 100 μM | 100 μM |
| | Disodium | Disodium |
| | Chromoglycate (DSCG) | Chromoglycate |
| | 100 μM | (DSCG) 100 μM |
| | Glycerol (>98%) | |
| | 0.69% (75 mM) | |

### Example 13

### Size-Based Separation of Fetal Cells from Maternal Blood/Composition G

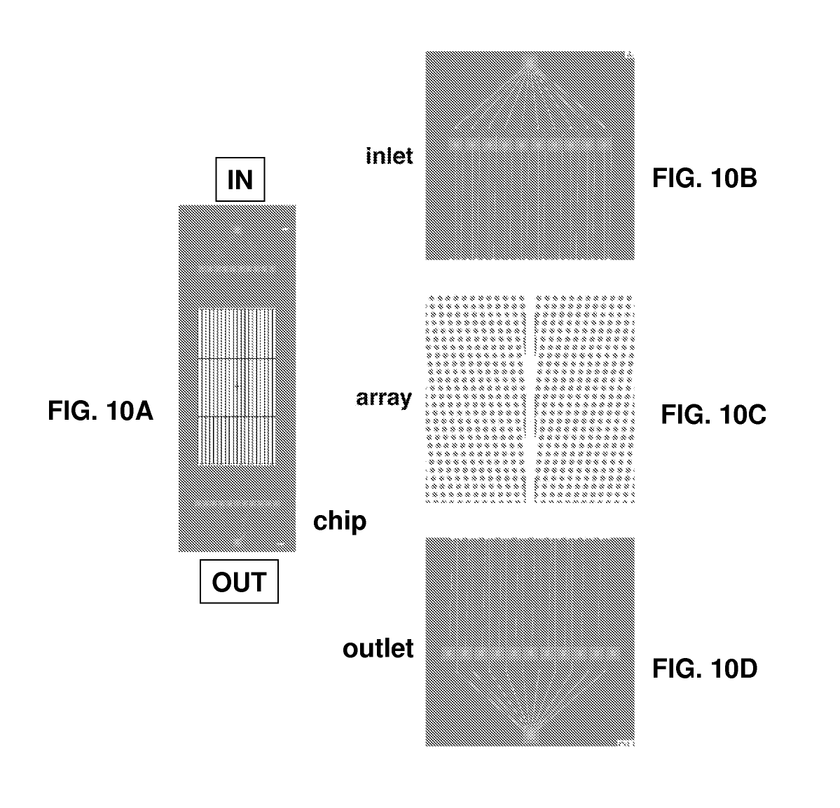

FIGS. 10A-10D shows a schematic of the device used to separate fetal nucleated red blood cells from maternal blood.

Dimensions: 100 mm×28 mm×1 mm

Array design: 3 stages, gap size 18, 12 and 8 μm for the first, second and third stage, respectively.

Device fabrication: The arrays and channels are fabricated in silicon using standard photolithography and deep silicon reactive etching techniques. The etch depth is 140 μm. Through holes for fluid access are made using KOH wet etching. The silicon substrate is sealed on the etched face to form enclosed fluidic channels using a blood compatible pressure sensitive adhesive (9795, 3M, St Paul, Minn.)

Device packaging: The device is mechanically mated to a plastic manifold with external fluidic reservoirs to deliver blood and buffer to the device and extract the generated fractions.

Device operation: An external pressure source is used to apply a pressure of 2.0 PSI to the buffer and blood reservoirs to modulate fluidic delivery and extraction from the packaged device.

Experimental conditions: Human maternal blood is drawn into a tube containing heparin and concentrated stabilization Composition G. Addition of the blood dilutes the stabilization composition to 1× concentration. 1 mL of blood/stabilization composition is processed at 3 mL/hr using the device described above at room temperature and within 48 hrs of draw. Nucleated cells from the blood are separated from enucleated cells (red blood cells and platelets), and plasma is delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100ML, Sigma-Aldrich, St Louis, Mo.) and 2 mM EDTA (15575-020, Invitrogen, Carlsbad, Calif.).

Measurement techniques: Cell smears of the product and waste fractions are prepared and stained with modified Wright-Giemsa (WG16, Sigma Aldrich, St. Louis, Mo.).

Performance: Fetal nucleated red blood cells are expected to be observed in the product fraction and absent from the waste fraction.

### Example 14

### Aneuploidy Determination by Sequencing Cell-Free DNA in Maternal Blood Mixed with Composition I

Cell-free fetal DNA in maternal blood can be analyzed to determine the presence or absence of fetal aneuploidy. Maternal blood is isolated and mixed with a composition such that the final concentrations of the components of the composition are that of Composition I (see Example 10).

Cell-free DNA is isolated from the maternal blood/Composition I mixture. Plasma or serum is obtained, and DNA from the plasma or serum is amplified by PCR. The amplified DNA is fragmented, and sheared ends are repaired and adenylated. Adapter oligos are ligated to both ends of the DNA fragments. The DNA fragments are hybridized to sequences complementary to the adapters on the surface of flow cell channels. The fragments are then bridge amplified, generating clusters of clonal fragments. The reverse strands are cleaved and removed. Ends are blocked, and a sequencing primer is hybridized to the templates. Clusters are sequenced simultaneously using 4 fluorescently labeled nucleotides. After each round of synthesis, the clusters are excited by a laser, emitting a color that identifies the base. The fluorescent label and blocking group are removed, allowing for the addition of the next base.

Chromosome fragments are enumerated to determine the presence or absence of trisomy 21 (Down syndrome). Sequences derived from maternal and fetal chromosome 21 and chromosome 1 are enumerated. Chromosome 21 is suspected of being trisomic in the fetal and euploid in the mother, and chromosome 1 is suspected of being euploid in the fetus and the mother. The ratio chromosome 21 fragments to chromosome 1 fragments is compared to values that would be expected if the fetus had trisomy 21 or if the fetus did not have trisomy 21. The presence or absence of trisomy 21 is determined.

### Example 15

### Density Gradient Centrifugation for Fetal Cell Enrichment

60% percoll (see Table 8) is made in Composition A. 1× Composition A Buffer (with 25 mM Hepes, 0.22% dextrose and 1% BSA, pH 7.2, Osmolarity 290) and PBS/1% BSA solutions are prepared, 500 ml each for one blood sample of 40 ml. Pool blood sample is pooled, and a 50 μl aliquot for CBC count is taken. 40 ml blood is aliquoted to 2×50 ml conical tubes, 1:1 diluted with 1× Composition A buffer. 20 ml 60% percoll solution is aliquoted in 4×50 ml conical tubes. 20 ml diluted blood is carefully overlayed on top of 20 ml percoll solution. The sample is spun at 1550 rpm for 30 min at RT, with the brake off. The plasma fraction is removed, leaving ˜1 ml above the buffy layer. The buffy layer is collected, leaving ˜500 μl above the red blood cell pellet. For each 10 ml whole blood, buffy layer product is split into 2×50 ml conical tubes, then 1×Composition A buffer is added up to 50 ml. The samples are spun at 1300 rpm for 10 min at RT, with the brake off. The supernatant is removed, and the pellet is gently resuspended into 1 ml PBS/1% BSA solution. The cells are pooled from 4 tubes into 1 tube. The volume is brought to 50 ml with PBS/1% BSA. The tube is spun at 1300 rpm for 10 min at RT, with brake off. The pellet is resuspended in 4 ml PBS/1% BSA for 40 ml whole blood equivalent and followed with CD71 selection. 50 μl CD71 is used for 10 ml whole blood equivalent.

TABLE 8
60% Percoll Solution

| 100 ml 60% percoll solution | ml |
|---|---|
| Percoll | 60 |
| 10x Composition A | 10 |
| 10x Hepes (250 mM), pH 7.0 | 10 |
| 1M NaCl | 8.5 |
| water | 11.5 |
| pH | 7.2-7.4 |
| Osmolarity | 280-290 |

### Example 16

### ACD Blood Anticoagulant Recipe

The following is a protocol for preparing ACD blood anticoagulant. In step 1, dissolve 1.32g of sodium citrate in 85 ml of distilled water. In step 2, dissolve 0.48 g of citric acid in the solution from step 1. In step 3, dissolve 1.47 g of dextrose in the solution from step 2. In step 4, add distilled water to 100 ml. In step 5, filter sterilize through 0.2 um filter. Use 0.25 ml of solution for 1 ml of blood (http://www.thelabrat.com/protocols/ACD.shtml).

### Example 17

### RBC Lysis Procedure with Composition Q

The following is a protocol for enriching a sample for intact fetal cells from maternal blood by selectively lysing enucleated red blood cells (RBC's) with a lysing reagent in combination with utilizing the fetal cell stabilization composition Composition Q.

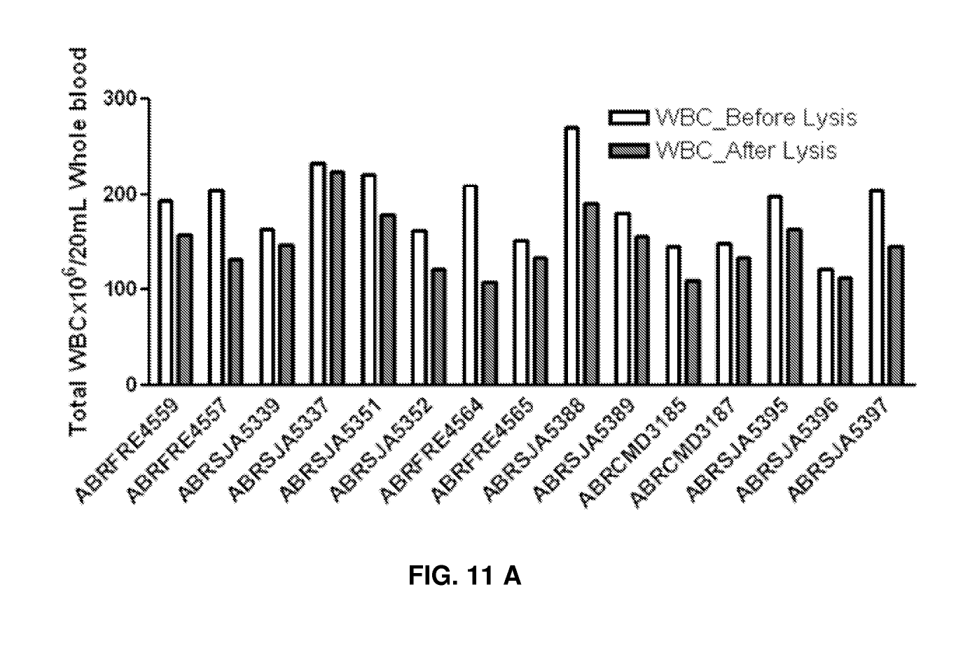

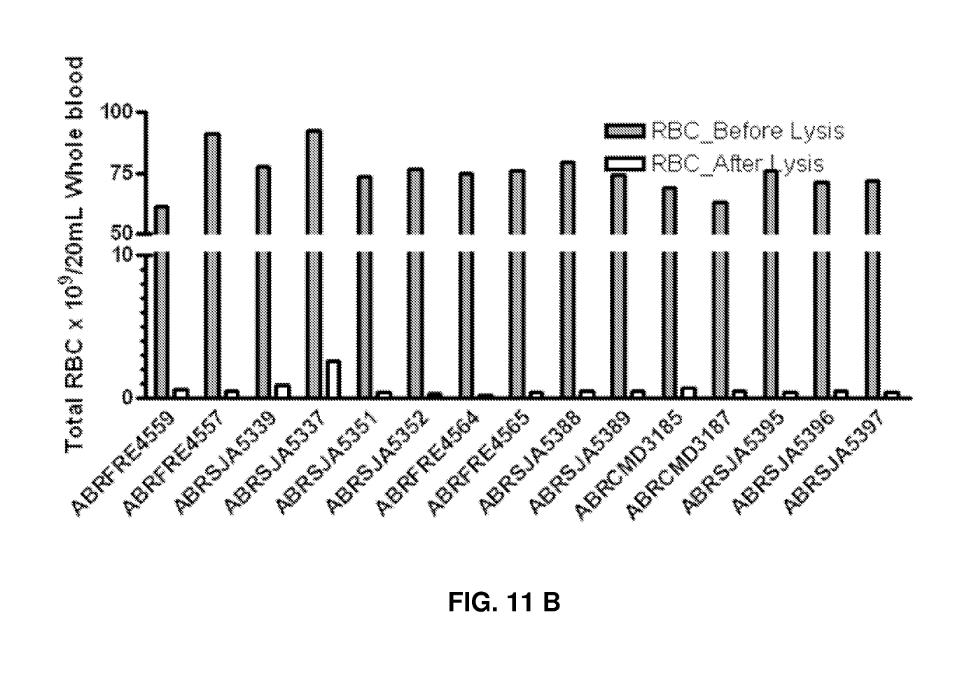

Maternal blood samples from 19 women carrying a male fetus (7-16 weeks of gestation) were treated with the lysing reagent HYL-250, and half of the samples were also treated with Composition Q (11 mM Sodium Citrate, 1.5 mL Theophylline, 0.37 mM Adenosine, 0.02 mM Dipyridamole, 0.25 mM Glycine, 0.25 mM NAC, 0.25 mM Glutamine, 0.04% Formaldehyde and 0.025% aged Potassium Dichromate) for 24 and 48 hours at room temperature. HYL-250 (Invitrogen, Carlsbad, Calif.) is a lysing reagent that selectively lyses enucleated RBC's (FIGS. 11A and B). Lysis of the samples was performed using a 8 parts HYL-250 to 1 part blood for 4 minutes with gentle shaking Nucleated cells including fetal cells that are not lysed by HYL-250 were harvested by centrifugation, washed once with phosphate buffered saline (PBS), and frozen. The cells were thawed, and genomic DNA was extracted using the Qiagen Maxiprep kit (Qiagen, Valencia, Calif.). The presence of fetal cells was determined by detecting Y-chromosome-specific DNA sequences using digital PCR. The PCR reaction was performed using two sets of primers and probe: the first primers and probe set identifies a first RPS4Y2 gene sequence on the Y-chromosome at nucleotide position 21,346,460, and the second primers and probe set identifies a second RPS4Y2 gene sequence on the Y-chromosome at nucleotide position 21,351,610. The forward and reverse primers, and the sequence of the probe for the first set were 5′-CCTCTCCCA ATCTCTACCAGGTATC (SEQ ID NO:1); 5′-AACCTCTGGCCTGGCTGACT (SEQ ID NO:2); and 5′-TACAGGGACGATGACTTT (SEQ ID NO:3), respectively. The forward and reverse primers, and the sequence of the probe for the second set were 5′-ATTTGGTACGTGAGAGATGATATG GT (SEQ ID NO:6), 5′-AACTATAGAGCTGCCAAGTGACACA (SEQ ID NO:4), and 5′-AAGCCTGCTGTTGCCT (SEQ ID NO:5). Cell-free DNA is believed to be about 300-500 bp in length, while isolated genomic DNA is typically detected as sequences of 10-20 kb. As the first and second primers and probe sequences are spaced by about 5 kb, detection of both primed genomic sequences is indicative of genomic DNA. The detection of both dPCR probes in the same well was termed “coincidental,” and was indicative of the presence of a fetal cell. The fetal cell count was normalized to account for PCR efficiency (0.85) to report a cell equivalent number for each sample, wherein: Cell Equivalent=Number of Coincidental Hits/0.85. Other target genes on the Y-chromosome DYS1 locus that can be used to detect fetal cells include the SMCY, EIF1AY, TTTY13, DAZ1, DAZ2, DAZ3 and DAZ4 genes.

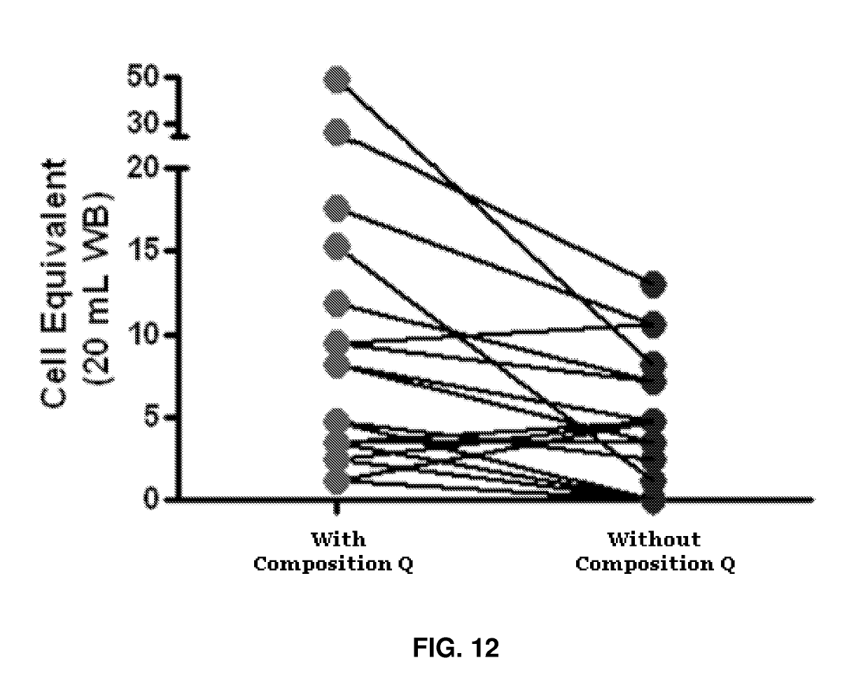

FIG. 12 shows the protective effect of Composition Q on fetal cell number. While all of the 19 samples (100%) that had been treated with Composition Q contained fetal cells, only 7 of the 19 samples (37%) that had not been treated with Composition Q contained fetal cells.

Thus, the data demonstrate that Composition Q effectively preserves fetal cells from a blood sample that is enriched by lysis of RBCs.

### Example 18

### Intact Fetal Cell Enrichment using Antibody-Based Enrichment

Maternal blood samples can be first enriched for fetal cells by one or more of methods utilizing size-based separation modules, density gradient centrifugation, and lysis of RBCs. The following is a protocol for a second enrichment of maternal samples that have previously undergone enrichment by selective lysis of RBCs as provided in Example 17.

A sample enriched for fetal cells by a first enrichment method using RBC lysis as described in Example 17, was spun at 1300 rpm for 10 minutes in a Beckman Allegra 6R centrifuge at 4° C. The supernatant was removed, leaving no more than 0.3 ml of liquid per 50 ml tube. The cell pellets were resuspended with 1 ml of 1% BSA/PBS buffer for every 10 ml of the original sample volume, and combined into one 50 ml tube. 200 μl of CD71 microbeads (Miltenyi Biotech, Cat No. 130-046-201) were added to the resuspended cells, and incubated on ice for 30 min while gently mixing using a VWR Rotator Waver (VWR Cat No. 12620-916) at Speed 4 and Tilt 8. The cells were then washed with 10 incubation volumes of 1% BSA/PBS buffer, and centrifuged at 1300 rpm for 10 minutes at 4° C. The cells were resuspended and applied to magnetic LS columns (Miltenyi Biotech, Cat No. 130-042-401) that had been previously rinsed with 3 ml of 1% BSA/PBS buffer.

CD71 negative cells contained in the column flowthrough were discarded. The column was washed three times with 5 ml of 1% BSA/PBS buffer, removed from its magnet, and placed on a 50 ml tube. 5 ml of 1% BSA/PBS buffer was used to elute the CD71-positive fetal cells coupled to the CD71 microbeads. Cytospin slides were made with the CD71-positive fetal cells for validation by immunocytochemistry (ICC). Fetal cells were first identified using a combination of a 1:400 dilution of rabbit monoclonal antibody to CK19 (ABCAM), 1:800 dilution of sheep anti-hemoglobin gamma (Bethyl), and a 1:100 dilution of a monoclonal antibody to fetal hemoglobin epsilon (Fitzgerald). The antibodies were visualized using a 1:250 dilution of a horse radish peroxidase (HRP) conjugated donkey anti-rabbit IgG (Jackson) using TSA-Plus Buffer (Perkin Elmer) Tyramide-Alexa 488 (Invitrogen). Antibody-positive fetal cells were further verified for the presence of Y-chromosome by DNA FISH analysis using X- and Y-chromosome probes, which respectively show blue and orange signals (Vysis, Abbott Molecular, Illinois).

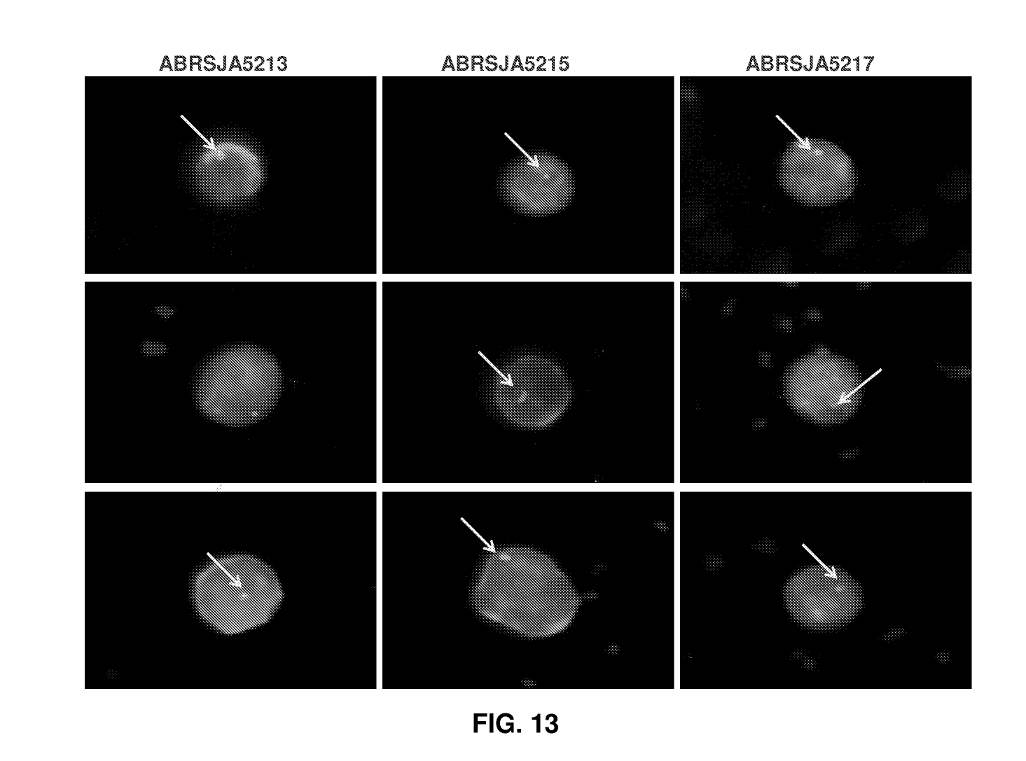

FIG. 13 shows the identification by ICC of fetal cells from three different samples (ABRSJA5213, ABRSJA5215, ABRSJA5217) following enrichment by RBC lysis and antibody-affinity enrichment using CD71 antibodies. The arrows point to the Y-chromosome identified by FISH. A number of fetal cell markers used for ICC are described in U.S. patent application Ser. No. 12/657,723. Cells that stained positive for fetal cell specific markers were enumerated.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A stabilization composition capable of maintaining at least 50% of fetal cells in a blood sample intact for at least 6 hr.

2. A stabilization composition capable of maintaining at least 50% of fetal nucleated red blood cells intact for at least 6 hr.

3. The composition of claim 1 or 2, wherein the composition is capable of maintaining at least 50% of fetal nucleated red blood cells intact for at least 12 hr, at least 24 hr, at least 48 hr, at least 72 hr, or at least 96 hr.

4. A composition comprising one or more isolated fetal cells in a stabilization composition of claim 1.

5. The composition of claim 1 or 2, wherein said composition is a solution.

6. A stabilization composition comprising:
four or more anticoagulants; and
two or more antioxidants.

7. The composition of claim 6, further comprising one or more of the following:
one or more energy sources;
one or more cell membrane stabilizers; and
one or more cross-linking agents.

8. A stabilization composition comprising:
two or more antioxidants; and
one or more cross-linking agents.

9. The composition of claim 8, further comprising one or more of the following:
one or more anticoagulants;
one or more energy sources; and
one or more cell membrane stabilizers.

10. A stabilization composition comprising: glycine, NAC, glutamine and D-Mannitol and optionally one or more anticoagulants, cell membrane stabilizers, or energy sources.

11. The composition of claim 1, 2, or 10, wherein said composition does not include (i) formaldehyde or (ii) an agent that slows cell metabolism.

12. The composition of claim 1, 2, or 6, wherein said composition does not include (i) potassium dichromate or (ii) a cell membrane stabilizing agent.

13. The composition of claim 6, 9, or 10, wherein said anticoagulant comprises at least one antiplatelet drug.

14. The composition of claim 13 wherein the at least one antiplatelet drug is selected from the group consisting of theophylline and dipyridamole.

15. The composition of claim 6, 9, or 10 wherein said anticoagulant comprises one or more of lithium heparin, sodium heparin, citrate heparin, ammonia heparin, sodium citrate, dipyridamole, theophylline, adenine, adenosine, Warfarin, acenocoumarol, phenindione, low molecular weight heparin, idraparinux, fondaparinux, argatroban, lepirudin, bivalirudin, and dabigatran.

16. The composition of claim 7, 9, or 10, wherein said energy source comprises glucose, lactose, fructose, or galactose.

17. The composition of claim 6 or 8, wherein said antioxidant comprises glycine, n-acetyl-L-cysteine, glutamine, D-Mannitol, vitamin C (ascorbic acid), vitamin E (tocopherols and tocotrienols), green tea, ferulic acid, reduced glutathione, melatonin, resveratrol, vitamin A (palmitate), beta carotene, vitamin D-3 (cholecalciferol), selenium (1-seleno methionine), BHA, or BHT.

18. The composition of claim 7, 9, or 10, wherein said cell membrane stabilizer comprises one or more of potassium dichromate, cadmium chloride, or lithium chloride aldehydes, urea formaldehyde, phenol formaldehyde, DMAE (dimethylaminoethanol), cholesterol, cholesterol derivatives, high concentrations of magnesium, vitamin E, and vitamin E derivatives, calcium, calcium gluconate, taurine, niacin, hydroxylamine derivatives, bimoclomol, sucrose, astaxanthin, glucose, amitriptyline, isomer A hopane tetral phenylacetate, isomer B hopane tetral phenylacetate, citicoline, inositol, vitamin B, vitamin B complex, cholesterol hemisuccinate, sorbitol, calcium, coenzyme Q, ubiquinone, vitamin K, vitamin K complex, menaquinone, zonegran, zinc, ginkgo biloba extract, diphenylhydantoin, perftoran, polyvinylpyrrolidone, phosphatidylserine, tegretol, PABA, disodium cromglycate, nedocromil sodium, phenyloin, zinc citrate, mexitil, dilantin, sodium hyaluronate, or polaxamer 188.

19. The composition of claim 7 or 8, wherein said cross-linking agent comprises one or more of formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfydryl addition or disulfide reduction, carbohydrate reactive crosslinkers, carboxyl reactive crosslinkers, photoreactive crosslinkers, cleavable crosslinkers, AEDP, APG, BASED, BM(PEO)₃, BM(PEO)₄, BMB, BMDB, BMH, BMOE, BS3, BSOCOES, DFDNB, DMA, DMP, DMS, DPDPB, DSG, DSP, DSS, DST, DTBP, DTME, DTSSP, EGS, HBVS, sulfo-BSOCOES, Sulfo-DST, or Sulfo-EGS.

20. The composition of claim 6, 8, or 10, wherein said composition further comprises one or more of PEG-200, PEG-300, PEG-400, PEG-600, PEG-1000, PEG-1450, PEG-3350, PEG-4000, PEG-6000, PEG-8000, PEG-20,000, imidazolidinyl urea, diazolidinyl urea, calcium propionate, sodium nitrate, sodium nitrite, sulfites, sulfur dioxide, sodium bisulfite, potassium hydrogen sulfite, disodium EDTA, ethanol, or methylchloroisothiazolinone.

21. The composition of claim 6, 8, or 10, wherein said composition further comprises a buffer.

22. The composition of claim 21, wherein said buffer comprises one or more of phosphate buffered saline (PBS), TAPS, Bicine, Tris, Tricine, HEPES, TES, MOPS, PIPES, Cacodylate, or MES.

23. A method for stabilizing a cell or cellular component comprising contacting said cell or cellular component with a composition of any one of claims 6-10.

24. The method of claim 23, wherein said cellular component is cell-free DNA.

25. The method of claim 23, wherein said cell is a fetal cell in a maternal blood sample.

26. A method for diagnosing a fetal condition comprising
contacting a maternal blood sample with a stabilization composition of any one of claims 6-10; and
analyzing one or more cells or cellular components from said sample to diagnosis said fetal condition.

27. The method of claim 26, further comprising enriching fetal cells from said sample using size-based separation, selective red blood cell lysis, or density gradient centrifugation.

28. The method of claim 26, further comprising contacting said sample with a lysis reagent that selectively lysis enucleated red blood cells over nucleated red blood cells.

29. The method of claim 26, further comprising performing an antibody-based enrichment step.

30. The method of claim 26, wherein said analyzing comprises performing fluorescent in-situ hybridization on DNA from said one or more cells or cellular components from said sample.

31. The method of claim 26, wherein said fetal condition comprises fetal aneuploidy.

32. The method of claim 31, wherein said aneuploidy comprises trisomy.

33. The method of claim 32, wherein said trisomy comprises trisomy 13, trisomy 18, or trisomy 21.

34. The method of claim 26, wherein said cellular component comprises cell-free DNA.

35. The method of claim 34, wherein said analyzing comprises DNA sequencing.

36. The method of claim 35, wherein said DNA sequencing comprises sequencing DNA from a first genomic region suspected of being trisomic and a second genomic region suspected of being aneuploid.

37. The method of claim 34, wherein said analyzing comprises digital PCR.

38. The method of claim 26, wherein said cell is a fetal nucleated red blood cell.

39. A test tube or syringe with a plug or a solution comprising a stabilization solution capable of maintaining at least 50% of fetal cells in a blood sample intact for at least 6 hr.

40. A test tube or syringe with a plug or a solution comprising a stabilization solution capable of maintaining at least 50% of fetal nucleated red blood cells in a blood sample intact for at least 6 hr.

41. The test tube or syringe of claim 39 or 40, wherein the composition is capable of maintaining at least 50% of fetal nucleated red blood cells intact for at least 12 hr, at least 24 hr, at least 48 hr, at least 72 hr, or at least 96 hr.

42. A test tube or syringe with a plug or a solution comprising a stabilization solution comprising:
four or more anticoagulants; and
two or more antioxidants.

43. The test tube or syringe of claim 42, further comprising one or more of the following:
one or more energy sources;
one or more cell membrane stabilizers; and
one or more cross-linking agents.

44. A test tube or syringe with a plug or a solution comprising a stabilization solution comprising:
two or more antioxidants; and
one or more cross-linking agents.

45. The test tube or syringe of claim 44, further comprising one or more of the following:
one or more anticoagulants;
one or more energy sources; and
one or more cell membrane stabilizers.

46. A test tube or syringe with a plug or a solution comprising a stabilization solution comprising: glycine, NAC, glutamine and D-Mannitol and optionally one or more anticoagulants, cell membrane stabilizers, or energy sources.

47. A kit comprising the test tube or syringe of claim 39, further comprising instructional material and materials for shipping a blood sample.
